(19) United States
(12) Patent Application Publication
(10) Pub. No.: US 2008/0109918 A1
(43) Pub. Date: May 8, 2008

(54) Materials and methods for tissue-specific targeting of ethylene insensitivity in transgenic plants

(76) Inventors: Harry J. Klee, Gainesville, FL (US); Coralie Lashbrook, Ames, IA (US); Lori Shrode, Germantown, MD (US)

Correspondence Address: ROGITZ & ASSOCIATES, 750 B STREET, SUITE 3120, SAN DIEGO, CA, 92101, US

(21) Appl. No.: 11/823,839

(22) Filed: Jun. 28, 2007

Related U.S. Application Data
(63) Continuation of application No. 10/602,475, filed on Jun. 23, 2003, now abandoned.
(60) Provisional application No. 60/390,385, filed on Jun. 21, 2002.

(51) Int. Cl. A01H 5/00; C07H 21/04; C12N 15/82; C12N 5/04

(52) U.S. Cl. 800/278; 435/410; 536/23.6; 800/298; 800/305; 800/306; 800/309; 800/310; 800/312; 800/313; 800/314; 800/315; 800/316; 800/317.1; 800/317.2; 800/317.3; 800/317.4; 800/320; 800/320.1; 800/320.2; 800/320.3; 800/323.2; 800/323.3

(57) ABSTRACT

The subject invention concerns materials and methods for controlling agricultural traits in plants that are mediated by the plant hormone ethylene. One aspect of the invention concerns a polynucleotide that comprises a sequence encoding a mutant ethylene receptor that is operably linked to a regulatory sequence that drives expression of the mutant receptor in a tissue-specific manner. In an exemplified embodiment, the mutant receptor sequence is an etr1-1 sequence, or a functional fragment or variant thereof, and the regulatory sequence is a promoter sequence from a cotton chitinase gene that can promote expression of the mutant ethylene receptor in abscission zone tissue of a plant. The subject invention also concerns plants and plant tissue transformed with the polynucleotide of the subject invention. Plants expressing the polynucleotide of the subject invention do not drop their flowers in response to exposure to ethylene.

### CROSS-REFERENCE TO RELATED APPLICATION

This application is a continuation of U.S. application Ser. No. 10/602,475, filed Jun. 23, 2003, which claims the benefit of U.S. Provisional Application Ser. No. 60/390,385, filed Jun. 21, 2002, the disclosure of each of which is hereby incorporated by reference in their entirety, including all figures, nucleic acid sequences, amino acid sequences, and tables.

### BACKGROUND OF INVENTION

Ethylene is a plant hormone associated with growth and development characteristics of a plant, including flower initiation, fruit, leaf and flower abscission, senescence, and fruit ripening. Abscission or shedding occurs when there is a separation of cells located in regions of a plant known as abscission zones. Regulation and control of ethylene production and action has long been a goal of plant biologists. Many of these are directed to controlling enzymes that are associated with the production of ethylene by the plant. More recently, efforts to control or modulate ethylene action have been directed to the plant receptor for ethylene. The gene sequence of an ethylene receptor in Arabidopsis thaliana, designated ETR1, can be found in Genbank (accession number L24119). ETR1 gene sequences, and the receptors encoded thereby, are known for other plants and include broccoli (Genbank accession no. AF047476); peach (Genbank accession no. AF124527); mango (Genbank accession no. AF227742); cucumber (Genbank accession no. AB026498); tobacco (Genbank accession no. AF022727); grape (Genbank accession no. AF243474); muskmelon (Genbank accession no. AB052228); tomato (Genbank accession no. AF043084); and tomato (Genbank accession no. U41103).

Some efforts have been directed to blocking the ethylene binding site of a plant ethylene receptor protein. Published U.S. patent application 20010019995 describes cyclopropene derivatives that bind to ethylene receptors and block the binding and activation by ethylene.

U.S. Pat. Nos. 6,294,716, 5,824,868, and 5,689,055 describe modified ethylene receptor wherein plants that express the receptor exhibit a decrease in their response to ethylene as compared to plants that are not expressing the modified receptor. However, all of the studies thus far modulating ethylene response using expression of a modified ethylene receptor in a plant have involved constitutive expression of the receptor throughout the tissues of the plant. It has been found that constitutive expression of the modified ethylene receptor leads to several unwanted side effects in the phenotypic characteristics of plants constitutively expressing the receptor. Accordingly, there remains a need in the art for means to modulate a plant's response to ethylene in a tissue-specific manner.

### BRIEF SUMMARY OF THE INVENTION

The subject invention concerns materials and methods for controlling agricultural traits in plants that are mediated by the plant hormone ethylene. Using the materials and methods of the invention, one can provide plants that are resistant to dropping their flowers, fruit, and/or leaves upon exposure to ethylene relative to wild type plants. One aspect of the invention concerns a polynucleotide that comprises a sequence encoding a mutant ethylene receptor that is operably linked to a regulatory sequence that drives expression of the mutant receptor in a tissue-specific manner. In an exemplified embodiment, the mutant receptor sequence is the Arabidopsis thaliana etr1-1 sequence, or a functional fragment or variant thereof, and the regulatory sequence is a promoter sequence from a cotton chitinase gene that can drive expression of the receptor specifically in an abscission zone of a plant.

The subject invention also concerns plants, plant tissue, and plant cells transformed with or bred to contain a polynucleotide of the subject invention. Plants expressing a polynucleotide of the subject invention do not drop their flowers in response to exposure to ethylene.

### BRIEF DESCRIPTION OF DRAWINGS

FIG. 1 shows a map of pLBS107 construct that includes a cotton chitinase promoter and mutant ethylene receptor that can be used according to the subject invention.

FIGS. 2A-2J show the sequence of the pLBS107 construct (SEQ ID NO: 9). The promoter sequence is approximately from nucleotide 1 to nucleotide 1622. The nucleotide sequence encoding the mutant ethylene receptor starts at nucleotide number 1674 and ends at nucleotide number 3887. The stop codon is located at nucleotides 3888-3890.

FIGS. 3A-3E show the sequence of the pLBS107 construct with the restriction sites identified over the nucleotide sequence.

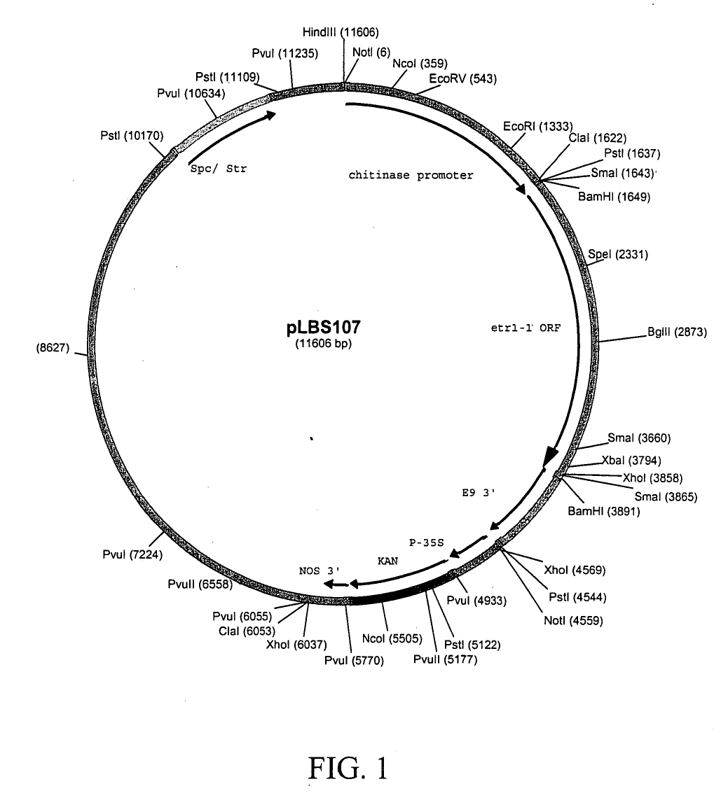

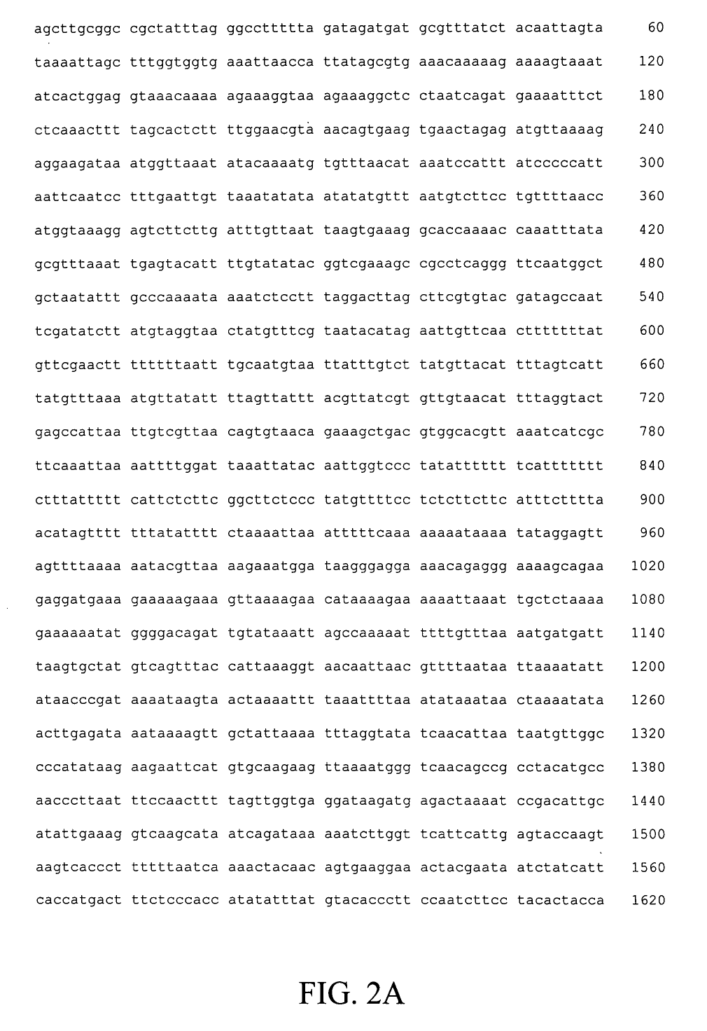

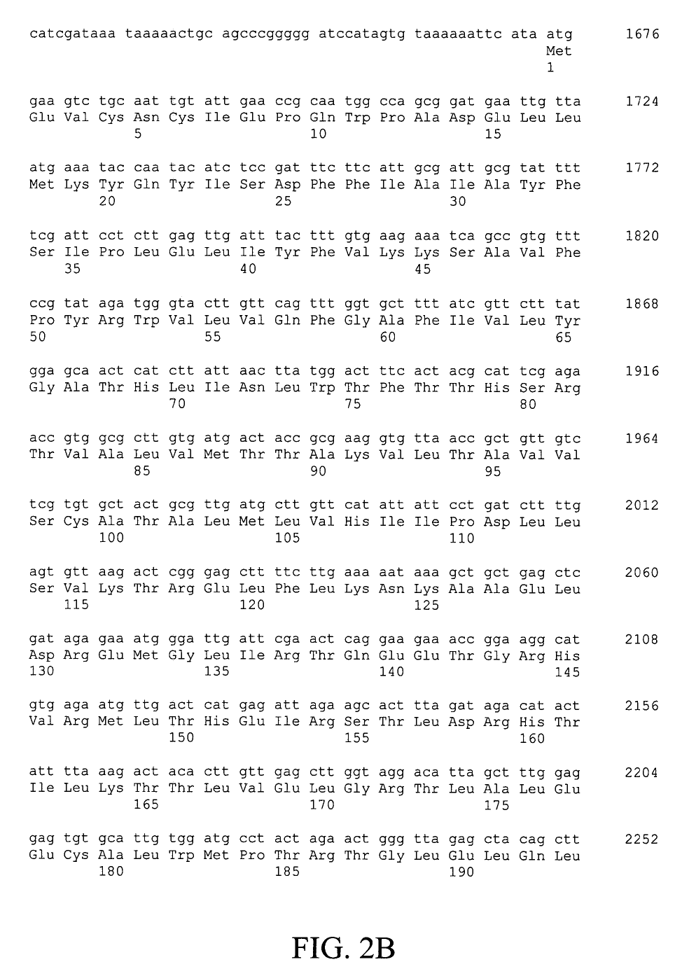

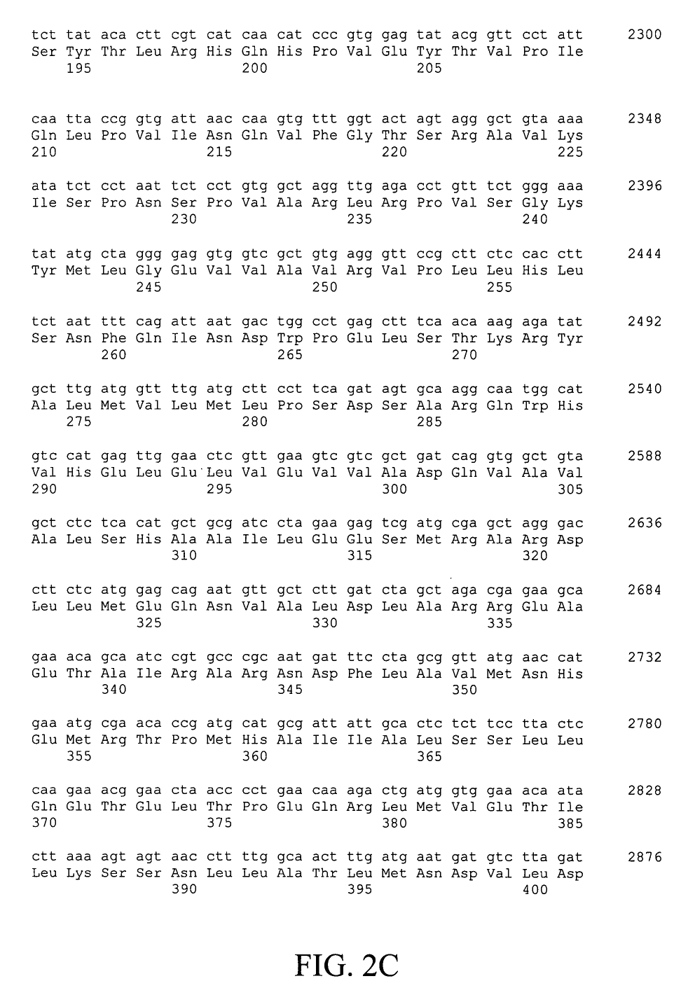

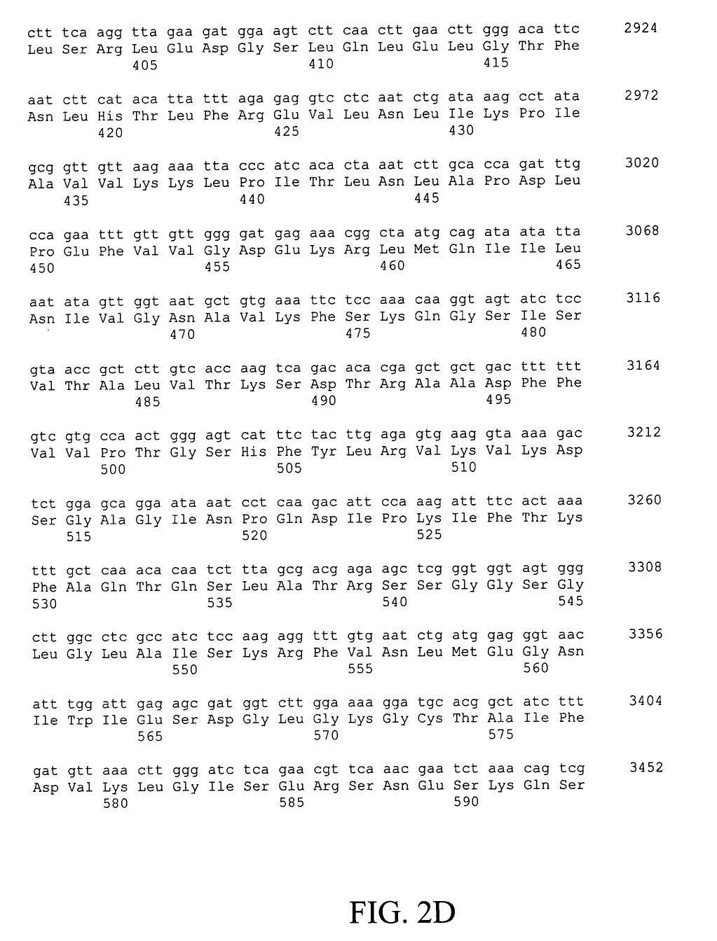

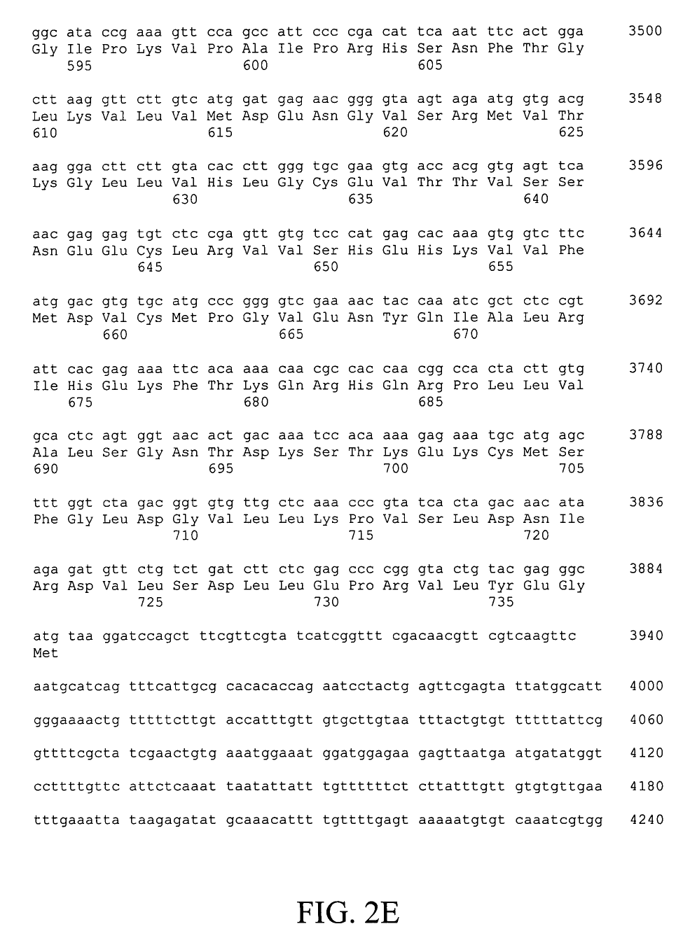

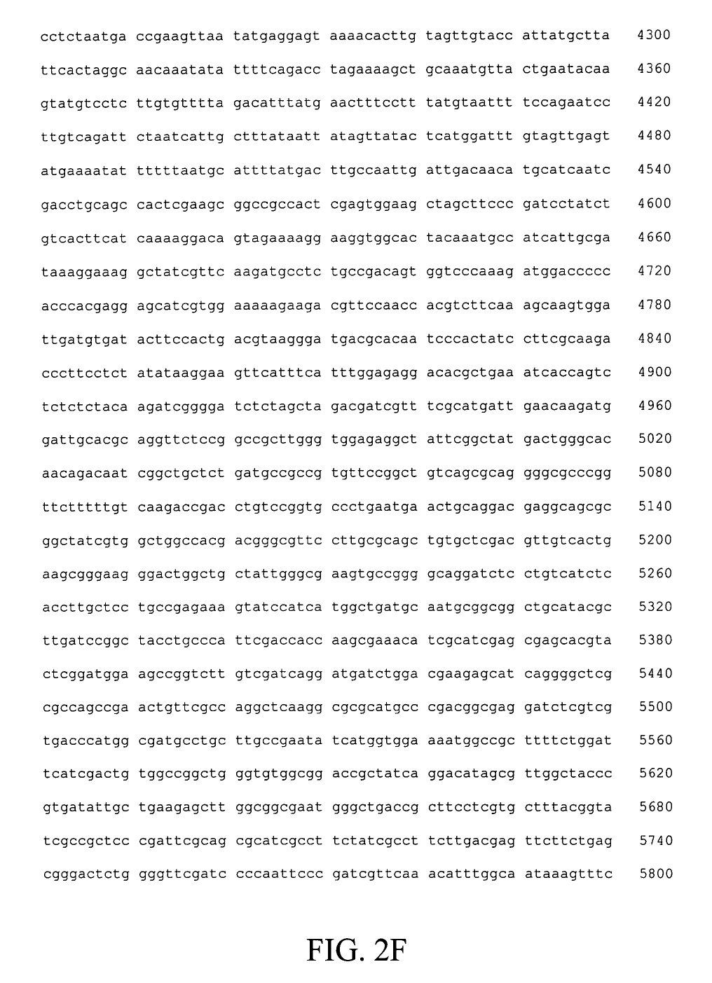

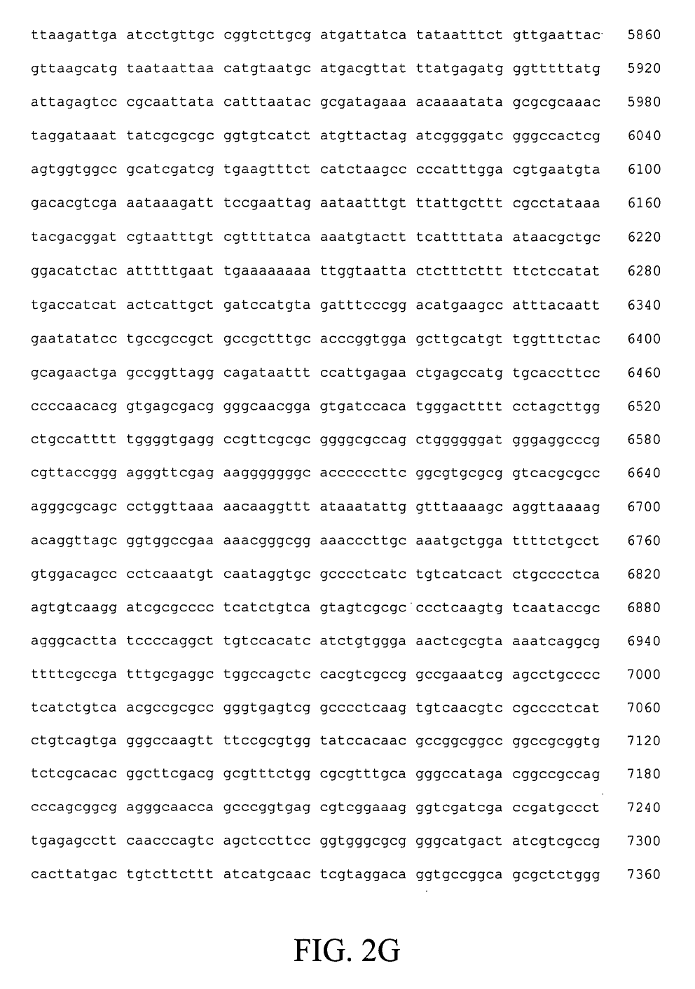

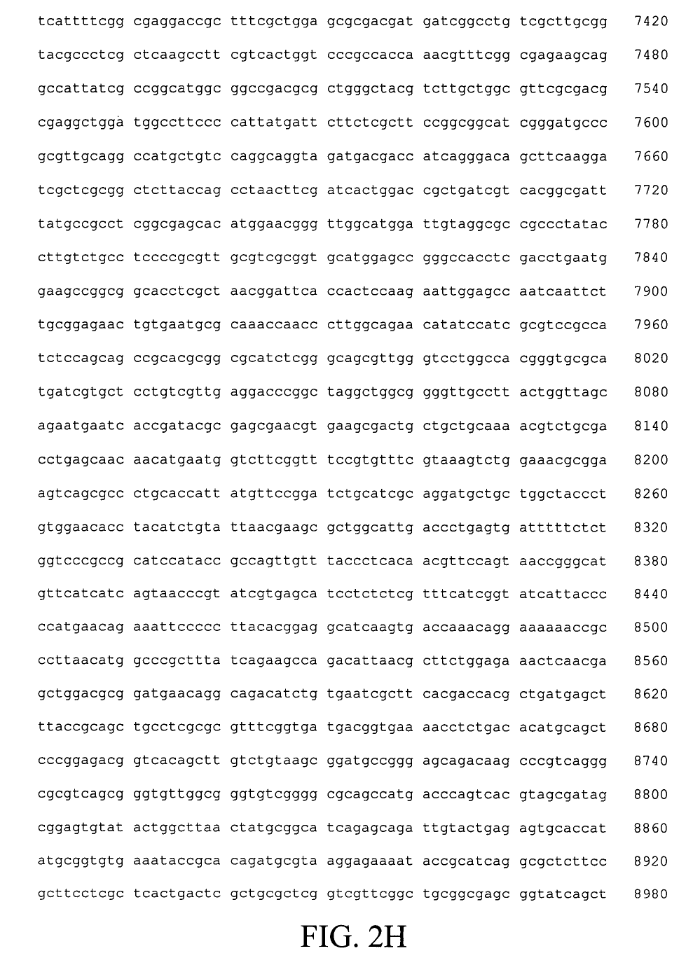

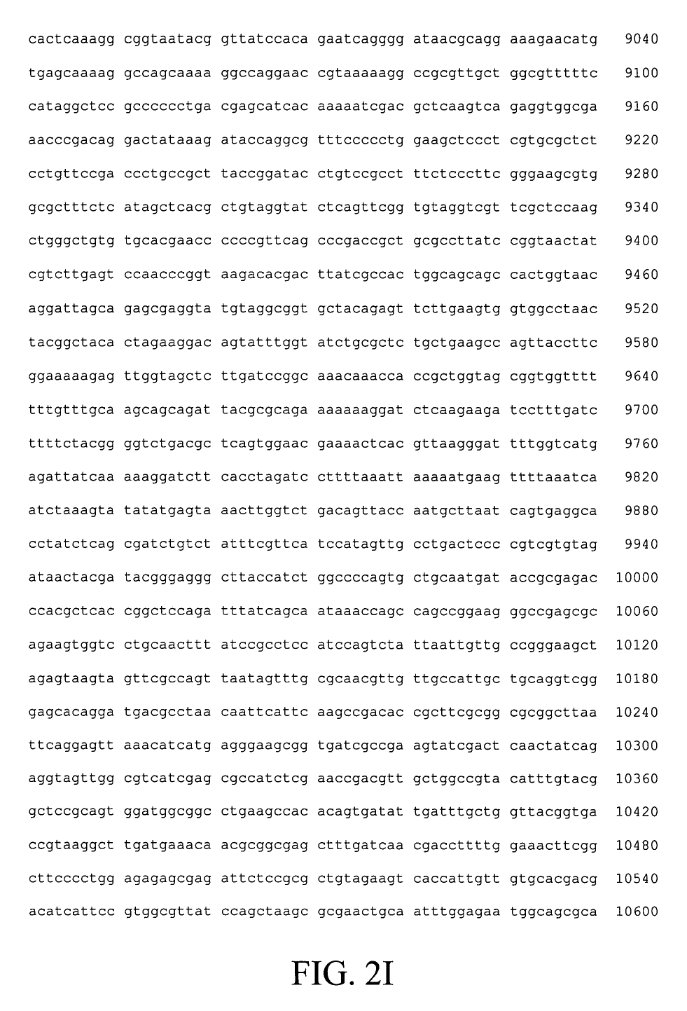

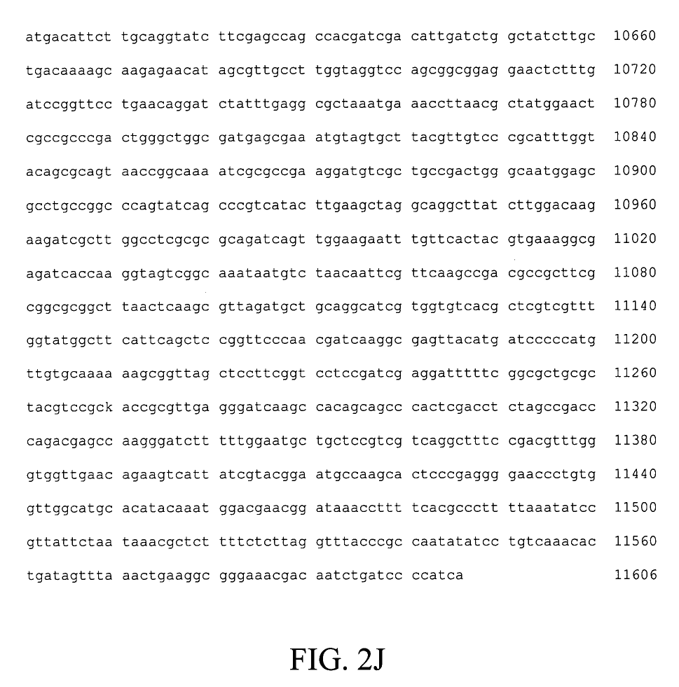

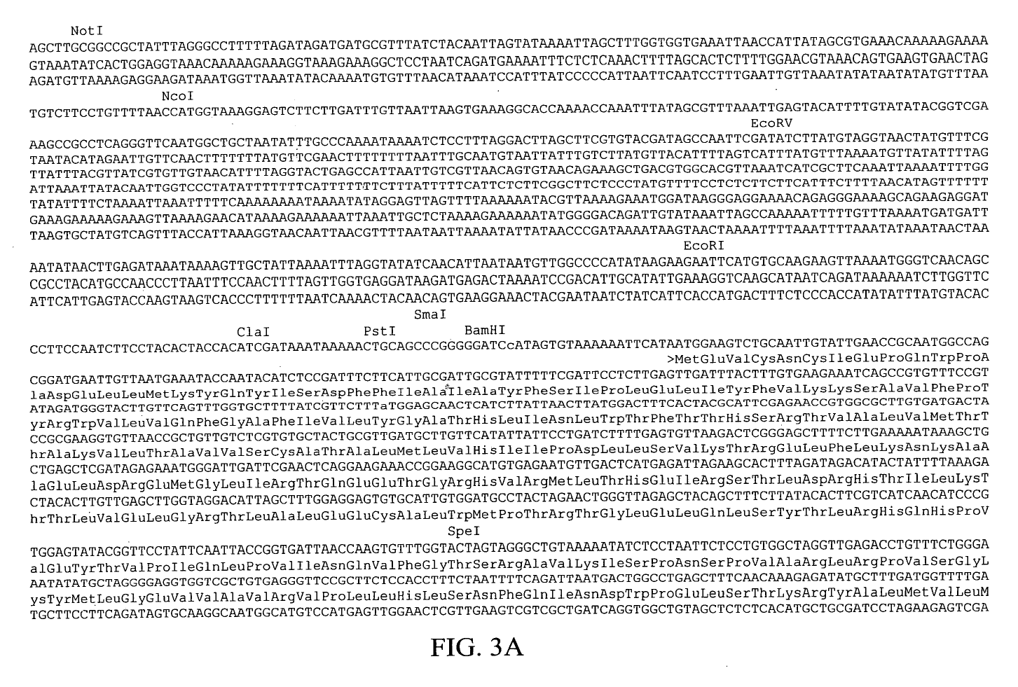

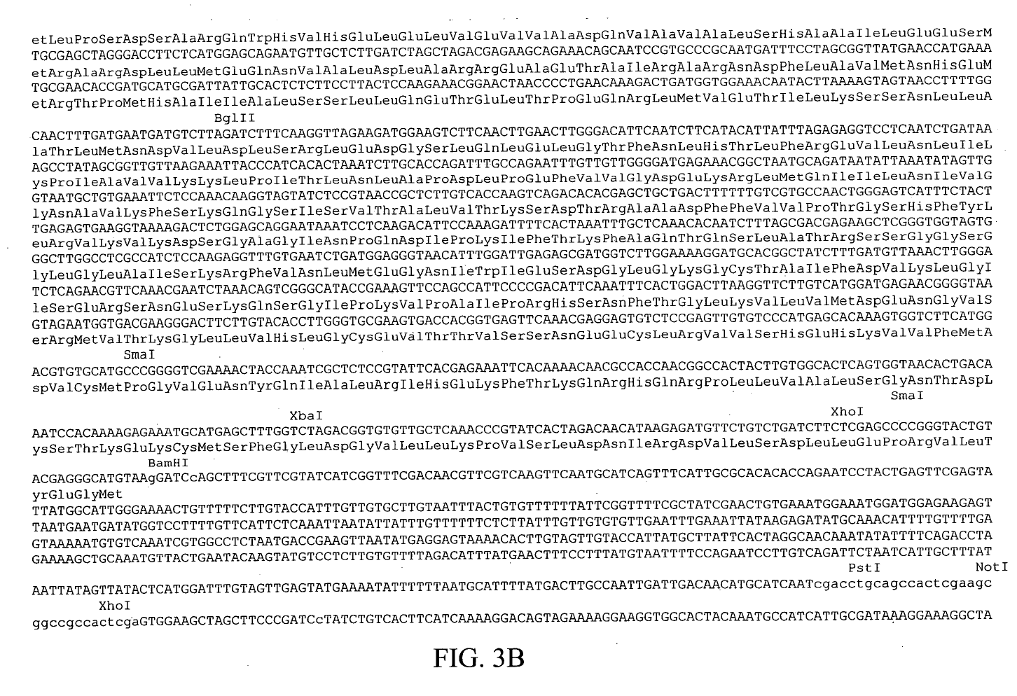

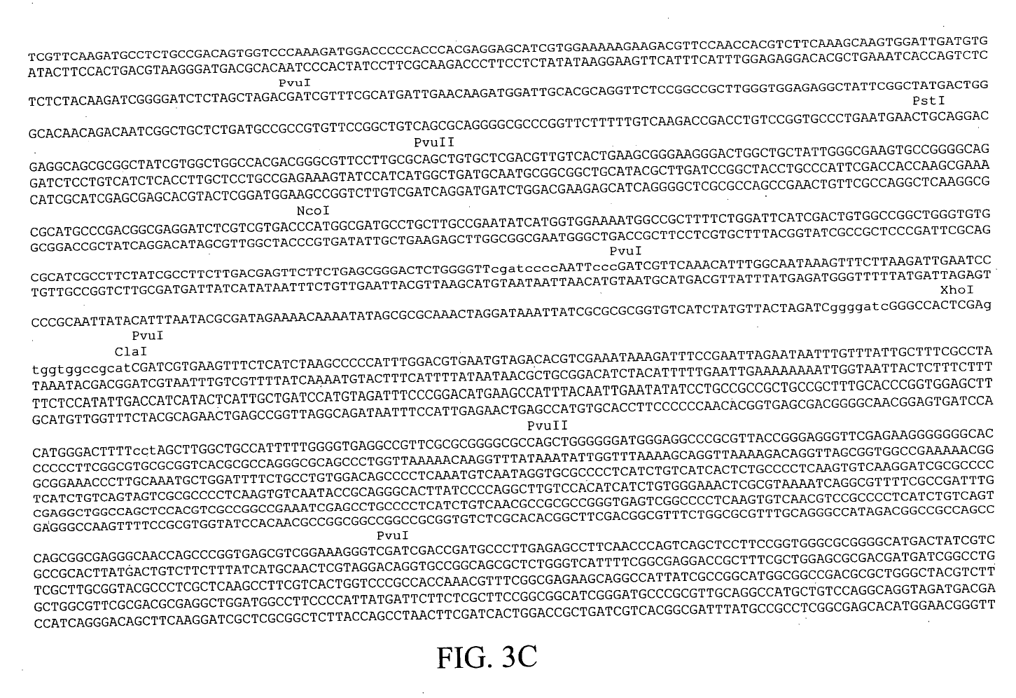

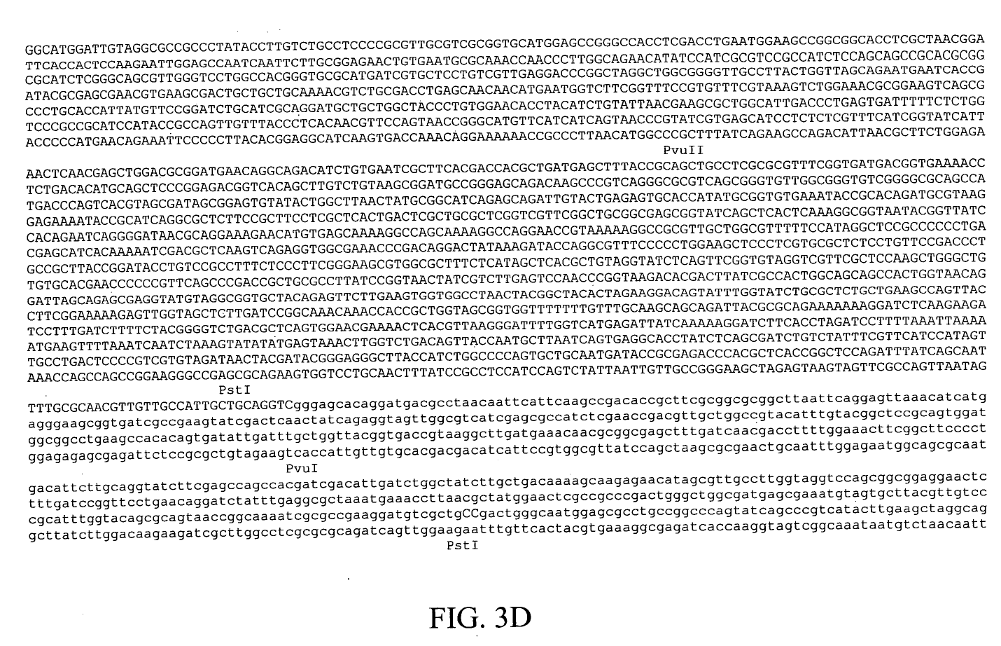

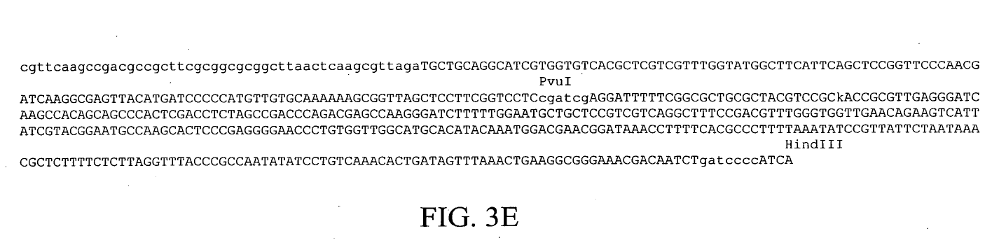

### BRIEF DESCRIPTION OF THE SEQUENCES

SEQ ID NO: 1 is an amino acid sequence of a mutant etr1-1 receptor that can be used according to the present invention.

SEQ ID NO: 2 is an amino acid sequence of a mutant etr1-2 receptor that can be used according to the present invention.

SEQ ID NO: 3 is an amino acid sequence of a mutant etr1-3 receptor that can be used according to the present invention.

SEQ ID NO: 4 is an amino acid sequence of a mutant etr1-4 receptor that can be used according to the present invention.

SEQ ID NO: 5 is a nucleotide sequence that comprises a protein coding sequence that encodes the mutant etr1-1 sequence shown as SEQ ID NO: 1 that can be used according to the present invention.

SEQ ID NO: 6 is an amino acid sequence of a mutant etr2-1 receptor that can be used according to the present invention.

SEQ ID NO: 7 is a nucleotide sequence that comprises a protein coding sequence that encodes the mutant etr2-1 sequence shown as SEQ ID NO: 6 that can be used according to the present invention.

SEQ ID NO: 8 is a cotton chitinase gene promoter sequence that can be used according to the present invention.

SEQ ID NO: 9 is the nucleotide sequence of the pLBS107 construct that can be used according to the present invention.

SEQ ID NO: 10 is a nucleotide sequence that comprises a sequence encoding an EIL1 polypeptide.

SEQ ID NO: 11 is a nucleotide sequence that comprises a sequence encoding an EIL2 polypeptide.

SEQ ID NO: 12 is a nucleotide sequence that comprises a sequence encoding an EIL3 polypeptide.

SEQ ID NO: 13 is a nucleotide sequence that comprises a sequence encoding an EIN2 polypeptide.

SEQ ID NO: 14 is a nucleotide sequence that comprises a sequence encoding an EIN2 polypeptide.

SEQ ID NO: 15 is a nucleotide sequence that comprises a sequence encoding an EIN3 polypeptide.

SEQ ID NO: 16 is a nucleotide sequence that comprises a sequence encoding an EIN3 polypeptide.

### DETAILED DISCLOSURE OF THE INVENTION

The subject invention concerns materials and methods for controlling agricultural traits in plants that are mediated by the plant hormone ethylene. One aspect of the invention concerns a polynucleotide that comprises: (a) a nucleotide sequence encoding a mutant plant ethylene receptor, or a fragment or variant thereof, that is ethylene insensitive, and (b) operably linked to the nucleotide sequence encoding the mutant receptor, a regulatory sequence that promotes transcription and expression of the nucleotide sequence encoding the mutant receptor in plant cells that comprise the abscission zone of a plant. Genes encoding plant ethylene receptors, and the amino acid sequences of the encoded proteins, have been identified and sequenced for numerous plant species. Plant ethylene receptors include those designated in the art as ETR1, ETR2, ERS1, ERS2, and EIN4 (Chang et al., 1993; Hua et al., 1995; Hua et al., 1998a; Hua et al. 1998b; and Sakai et al., 1998). ETR2 receptor sequences, and polynucleotides encoding them, are known for several plant species and include Arabidopsis thaliana (Genbank accession no. AF047975); cucumber (Genbank accession no. AB026500); apple tree (Genbank accession no. AF032448); and tomato (Genbank accession no. AF043085). Mutant alleles of these ethylene receptors that exhibit dominant insensitivity to ethylene have also been isolated and sequenced. Any polynucleotide sequence encoding a mutant ethylene receptor, or a fragment or variant thereof, that confers insensitivity to ethylene when expressed in a plant is contemplated within the scope of the present invention. As used herein, the term “operably linked” refers to a juxtaposition of the components described wherein the components are in a relationship that permits them to function in their intended manner. In general, operably linked components are in contiguous relation.

In one embodiment, the mutant receptor sequence can have an amino acid sequence corresponding to a plant etr1 mutant receptor. In a further embodiment, the mutant receptor sequence is an Arabidopsis thaliana etr1 sequence that can include, but is not limited to, etr1-1 (SEQ ID NO: 1), etr1-2 (SEQ ID NO: 2), etr1-3 (SEQ ID NO: 3), and etr1-4 (SEQ ID NO: 4) (Chang et al., 1993). In an exemplified embodiment, the mutant receptor sequence is the Arabidopsis thaliana etr1-1 sequence (SEQ ID NO: 1) in which the mutant form has a tyrosine in place of the cysteine at amino acid position 65 of the wildtype form, or a functional fragment or variant thereof, and the mutant ethylene receptor etr1-1 is encoded by the etr1-1 nucleotide sequence shown in SEQ ID NO: 5. In another embodiment, the mutant receptor sequence can have an amino acid sequence corresponding to a plant etr2 mutant receptor. The Arabidopsis thaliana ethylene receptor etr2-1 (SEQ ID NO: 6) is encoded by the etr2-1 gene sequence (SEQ ID NO: 7). Any nucleotide sequence that encodes a mutant ethylene receptor sequence of the present invention is contemplated within the scope of the invention.

Another aspect of the invention concerns materials and methods for inhibiting or reducing expression of genes that are involved in the ethylene signaling pathway in plants. These genes include, but are not limited to, EIN2, EIN3, and EIN3-like (EIL) genes (Alonso et al., 1999; Chao et al., 1997; Tieman et al., 2001 (SEQ ID NO: 10; SEQ ID NO: 11; and SEQ ID NO: 12); Genbank accession nos. NM120406 (SEQ ID NO: 13) and AF141202 (SEQ ID NO: 14); Genbank accession nos. NM112968 (SEQ ID NO: 15) and AF004216 (SEQ ID NO: 16)). Antisense, cosuppression, RNA interference (RNAi), and gene mutagenesis technologies can be used to inhibit expression or function of EIN or EIL genes or gene products. Polynucleotides that provide for transcribed nucleic acid sequences that are at least partially complementary to a transcribed sequence of an EIN or EIL gene are contemplated within the scope of the invention. Such polynucleotides are referred to herein as antisense polynucleotides and the sequences are antisense sequences. Transcription of the antisense sequence results in production of RNA which is at least partially complementary to RNA transcribed from an EIN or EIL gene. The polynucleotide does not have to be identical in sequence to or the same length as the endogenous EIN or EIL gene sequence. The polynucleotide used for antisense inhibition can be shorter in length than the full-length EIN or EIL sequence. For example, a polynucleotide can be used that corresponds to the 5′-end or the 3′-end of the endogenous EIN or EIL gene. The polynucleotide sequence that is complementary to a sequence of an mRNA of an EIN or EIL gene is selected to be of sufficient length to bind to the mRNA and inhibit expression of the gene product. The sequence is preferably between 10 and 5000 nucleotides in length. More preferably, the sequence is between 20 and 2000 nucleotides in length. Most preferably, the sequence is between 50 and 1000 nucleotides in length. The sequence transcribed from the antisense polynucleotide may be complementary to any sequence of the RNA transcribed from an EIN or EIL gene, including the 5′ non-coding sequence, 3′ non-coding sequence, introns, the coding sequence, or any portion thereof.

Inhibition of expression of an endogenous EIN or EIL gene can also be achieved by introducing into a plant cell a polynucleotide comprising a nucleotide sequence that is identical to or similar to the sequence of an endogenous EIN or EIL gene sequence, and selecting from among transformed plants obtained from the cells those transformants that express the EIN or EIL transgene sequence and that exhibit reduced expression of EIN or EIL gene products as compared to non-transformed plants. This method of inhibiting expression of a gene product is also referred to as RNAi, cosuppression, or “post-transcriptional gene silencing.” The polynucleotide does not have to be identical in sequence to or the same length as the endogenous gene sequence. The polynucleotide can be shorter in length than the full-length gene sequence. For example, a polynucleotide can be used that corresponds to the 5′-end or the 3′-end of the endogenous gene. Other methods for inhibiting expression of a gene product through RNAi, cosuppression, or post-transcriptional gene silencing are known in the art. For example, an expression vector that provides for the continual expression of small interfering RNAs (siRNAs) in transiently or stably transfected cells can be used. The siRNAs are small double-stranded RNAs (dsRNAs) of 21-23 nucleotides. The siRNAs comprise RNA sequences that are complementary to the sense and antisense strands of the gene that is being silenced. Expression of these double-stranded RNAs in a cell results in the inhibition of expression of the gene product. Polynucleotide sequences used to inhibit expression of an EIN or EIL gene product can have operably linked thereto a regulatory sequence that promotes transcription and expression of the nucleotide sequences used to inhibit EIN and EIL expression in plant cells that comprise the abscission zone of a plant.

In one embodiment, a regulatory sequence that can be operably linked to a nucleotide sequence of the present invention comprises a promoter from a plant chitinase gene, or a functional fragment or variant thereof. In an exemplified embodiment, the chitinase promoter is from cotton and comprises the nucleotide sequence shown in SEQ ID NO: 8, or a functional fragment or variant thereof. U.S. Pat. No. 5,399,680 describes a promoter from rice chitinase that can be used with the invention. Other promoters that can drive expression in cells comprising the abscission zones of a plant are also contemplated within the scope of the invention and include, for example, promoter sequences of plant genes encoding polygalacturonases or cellulases (Koehler et al., 1996; Genbank accession no. U34754; Genbank accession no. U34755). As used herein, “promoter” or “promoter sequence” means a polynucleotide sequence of a nucleic acid molecule that is capable of directing an RNA polymerase to initiate transcription (i.e., the synthesis of RNA on a DNA template) at a transcription initiation site.

A promoter sequence can be incorporated into a polynucleotide of the invention using standard techniques known in the art. Multiple copies of promoters or multiple promoters can be used in an expression construct of the invention. Typically, a promoter sequence is operably linked 5′ to the nucleotide sequence encoding a mutant ethylene receptor. In one embodiment, a promoter can be positioned about the same distance from the transcription start site as it is from the transcription start site in its natural genetic environment. Some variation in this distance is permitted without substantial decrease in promoter activity. A transcription start site is typically included in the expression construct.

The polynucleotide of the invention can include additional regulatory elements, for example, transcription termination sequences, translation termination sequences, enhancers, and polyadenylation elements.

A polynucleotide of the invention may optionally contain a transcription termination sequence, a translation termination sequence, a sequence encoding a signal peptide sequence and/or enhancer elements. Transcription termination regions can typically be obtained from the 3′ untranslated region of a eukaryotic or viral gene sequence. Transcription termination sequences can be positioned downstream of a coding sequence to provide for efficient termination. Signal peptides are a group of short amino terminal sequences that encode information responsible for the relocation of an operably linked mature polypeptide to a wide range of post-translational cellular destinations, ranging from a specific organelle compartment to sites of protein action and the extracellular environment. Targeting gene products to an intended cellular and/or extracellular destination through the use of operably linked signal peptide sequence is contemplated for use with the polypeptides of the invention. Enhancers are cis-acting elements that increase activity of a promoter and can also be included in the expression construct. Enhancer elements are known in the art, and include, but are not limited to, the CaMV 35S enhancer element, maize shrunken-1 enhancer element, cytomegalovirus (CMV) early promoter enhancer element, and the SV40 enhancer element.

DNA sequences which direct polyadenylation of mRNA transcribed from a polynucleotide can also be included in a polynucleotide of the invention, and include, but are not limited to, an octopine synthase or nopaline synthase signal. A polynucleotide of the invention can also include a polynucleotide sequence that directs transposition of other genes, i.e., a transposon.

A polynucleotide of the invention can also include one or more dominant selectable marker genes, including, for example, genes encoding antibiotic resistance and/or herbicide-resistance for selecting transformed cells. Antibiotic-resistance genes can provide for resistance to one or more of the following antibiotics: hygromycin, kanamycin, bleomycin, G418, streptomycin, paromomycin, neomycin, and spectinomycin. Kanamycin resistance can be provided by neomycin phosphotransferase (NPT II). Herbicide-resistance genes can provide for resistance to phosphinothricin acetyltransferase or glyphosate. Other markers used for cell transformation screening include genes encoding β-glucuronidase (GUS), β-galactosidase, luciferase, nopaline synthase, chloramphenicol acetyltransferase (CAT), green fluorescence protein (GFP), or enhanced GFP (Yang et al., 1996).

The subject invention also concerns polynucleotide vectors comprising a polynucleotide sequence of the invention. Unique restriction enzyme sites can be included at the 5′ and 3′ ends of an expression construct or polynucleotide of the invention to allow for insertion into a polynucleotide vector. As used herein, the term “vector” refers to any genetic element, including for example, plasmids, cosmids, chromosomes, phage, virus, and the like, which is capable of replication when associated with proper control elements and which can transfer polynucleotide sequences between cells. Vectors contain a nucleotide sequence that permits the vector to replicate in a selected host cell. A number of vectors are available for expression and/or cloning, and include, but are not limited to, pBR322, pUC series, M13 series, and pBLUESCRIPT vectors (Stratagene, La Jolla, Calif.).

Polynucleotides of the present invention can be composed of either RNA or DNA. Preferably, the polynucleotides are composed of DNA. The subject invention also encompasses those polynucleotides that are complementary in sequence to the polynucleotides disclosed herein.

Because of the degeneracy of the genetic code, a variety of different polynucleotide sequences can encode a mutant receptor disclosed herein. In addition, it is well within the skill of a person trained in the art to create alternative polynucleotide sequences encoding the same, or essentially the same, polypeptides of the subject invention. These variant or alternative polynucleotide sequences are within the scope of the subject invention. As used herein, references to “essentially the same” sequence refers to sequences which encode amino acid substitutions, deletions, additions, or insertions which do not materially alter the functional activity of the polypeptide encoded by the polynucleotides of the present invention.

Mutant ethylene receptor proteins having substitution of amino acids other than those specifically exemplified in the subject receptor proteins are also contemplated within the scope of the present invention. For example, non-natural amino acids can be substituted for the amino acids of a protein of the invention, so long as the protein having substituted amino acids retains substantially the same activity as the protein in which amino acids have not been substituted. Examples of non-natural amino acids include, but are not limited to, ornithine, citrulline, hydroxyproline, homoserine, phenylglycine, taurine, iodotyrosine, 2,4-diaminobutyric acid, α-amino isobutyric acid, 4-aminobutyric acid, 2-amino butyric acid, γ-amino butyric acid, ε-amino hexanoic acid, 6-amino hexanoic acid, 2-amino isobutyric acid, 3-amino propionic acid, norleucine, norvaline, sarcosine, homocitrulline, cysteic acid, τ-butylglycine, τ-butylalanine, phenylglycine, cyclohexylalanine, β-alanine, fluoro-amino acids, designer amino acids such as β-methyl amino acids, C-methyl amino acids, N-methyl amino acids, and amino acid analogues in general. Non-natural amino acids also include amino acids having derivatized side groups. Furthermore, any of the amino acids in the protein can be of the D (dextrorotary) form or L (levorotary) form.

Amino acids can be generally categorized in the following classes: non-polar, uncharged polar, basic, and acidic. Conservative substitutions whereby a protein having an amino acid of one class is replaced with another amino acid of the same class fall within the scope of the subject invention so long as the protein having the substitution still retains substantially the same biological activity as a protein that does not have the substitution. Table 1 below provides a listing of examples of amino acids belonging to each class.

TABLE 1

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

The subject invention also concerns polynucleotides that encode mutant ethylene receptors of the invention. Because of the degeneracy of the genetic code, a variety of different polynucleotide sequences can encode a protein of the present invention. In addition, it is well within the skill of a person trained in the art to create alternative polynucleotide sequences encoding the same, or essentially the same, proteins of the subject invention. These variant or alternative polynucleotide sequences, and the proteins encoded thereby, are within the scope of the subject invention. As used herein, references to “essentially the same” sequence refers to sequences which encode amino acid substitutions, deletions, additions, and/or insertions which do not materially alter the functional activity of the protein encoded by the polynucleotides of the present invention. Variant proteins having amino acid substitutions, deletions, additions, and/or insertions which do not materially alter the functional activity of the mutant receptor protein can also be prepared using standard techniques known in the art, and such variant proteins are encompassed within the scope of the present invention. Polynucleotide sequences encoding a protein of the invention can be selected based on preferred codon usage of the organism in which it will be expressed. For example, the polynucleotide sequence can be selected for preferred codon usage in plant cells.

Polynucleotides and proteins of the subject invention can also be defined in terms of more particular identity and/or similarity ranges with those exemplified herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein. Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

The subject invention also contemplates those polynucleotide molecules having sequences which are sufficiently homologous with the polynucleotide sequences exemplified herein so as to permit hybridization with that sequence under standard stringent conditions and standard methods (Maniatis, T. et al., 1982). As used herein, “stringent” conditions for hybridization refers to conditions wherein hybridization is typically carried out overnight at 20-25 C below the melting temperature (Tm) of the DNA hybrid in 6×SSPE, 5×Denhardt's solution, 0.1% SDS, 0.1 mg/ml denatured DNA. The melting temperature is described by the following formula (Beltz, G. A. et al., 1983):
Tm=81.5C+16.6 Log [Na+]+0.41(% G+C)−0.61(% formamide)-600/length of duplex in base pairs.

Washes are typically carried out as follows:

(1) Twice at room temperature for 15 minutes in 1×SSPE, 0.1% SDS (low stringency wash).

(2) Once at Tm-20 C for 15 minutes in 0.2×SSPE, 0.1% SDS (moderate stringency wash).

As used herein, the terms “nucleic acid” and “polynucleotide sequence” refer to a deoxyribonucleotide or ribonucleotide polymer in either single- or double-stranded form, and unless otherwise limited, would encompass known analogs of natural nucleotides that can function in a similar manner as naturally-occurring nucleotides. The polynucleotide sequences include both the DNA strand sequence that is transcribed into RNA and the RNA sequence that is translated into protein. The polynucleotide sequences include both full-length sequences as well as shorter sequences derived from the full-length sequences. It is understood that a particular polynucleotide sequence includes the degenerate codons of the native sequence or sequences which may be introduced to provide codon preference in a specific host cell. Allelic variations of the exemplified sequences also come within the scope of the subject invention. The polynucleotide sequences falling within the scope of the subject invention further include sequences which specifically hybridize with the exemplified sequences. The polynucleotide includes both the sense and antisense strands as either individual strands or in the duplex.

In one embodiment of the subject method, a plant, plant tissue, or plant cell is transformed with a polynucleotide of the present invention and a transformed plant comprising the polynucleotide is grown from the transformed cell, tissue or plant.

Plants, plant tissue, and plant cells bred to contain or transformed with the mutant polynucleotides of the invention, and expressing the polypeptides encoded by the polynucleotides, are also contemplated by the present invention. Plants expressing a polynucleotide of the subject invention do not drop their flowers in response to exposure to ethylene. Plants within the scope of the present invention include monocotyledonous plants, such as rice, wheat, barley, oats, rye, sorghum, maize, lilies, banana, pineapple, turfgrass, gladiolus, and millet, and dicotyledonous plants, such as cotton, peas, alfalfa, chickpea, chicory, clover, kale, lentil, prairie grass, soybean, tobacco, potato, sweet potato, radish, cabbage, rape, apple trees, coffee, tomato, melon, citrus, beans, roses, sugar beet, squash, peppers, strawberry, carnation, chrysanthemums, impatiens, eucalyptus, and lettuce. In a particularly preferred embodiment, the plant is cotton or an ornamental plant such as lily, carnation, chrysanthemum, petunia, rose, geranium, orchid, gladioli, daisy, and tulip. Techniques for transforming plants with a gene are known in the art and include, for example, Agrobacterium infection, biolistic methods, etc.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1

### Results from Transformants Containing PLBS105

Plants transformed with plasmid pLBS105, which contains a promoter from a cotton chitinase gene fused to beta-glucuionidase reporter gene, were produced by standard Agrobacterium-mediated transformation as described in McCormick et al. (1986). Twelve independent transformation events were obtained. Of these lines, eleven were shown to express the reporter gene in the target tissue, flower abscission zones. The majority of these expressed the reporter gene at high levels. Only a subset of these lines (two) showed absolute specificity in expression, with the others exhibiting weak non-specific expression in other tissues. This result illustrates the difficulty of identifying transcriptional promoters that are completely tissue-specific. However, total tissue specificity is not necessarily required for success of the invention, as weak non-specific expression may not be sufficient to cause negative phenotypic effects in the non-target tissues.

### EXAMPLE 2

### Results from Transformants Containing PLBS107

Plants were transformed with plasmid pLBS107 (SEQ ID NO. 9), which contains a promoter from a cotton chitinase gene fused to the ethylene receptor gene etr1-1 and regenerated as above. The etr1-1 gene product, when expressed in plant tissues, results in ethylene insensitivity in those tissues. To date, a total of 39 independent transgenic events have been generated. Of these independent lines, 17 have been evaluated for their ability to retain flowers under stress conditions (Table 2). The stress involved treating intact tomato plants for 48 hours with 10 parts per million ethylene gas in a closed container. The chamber was opened after 16 hours to permit exchange of fresh air, resealed and ethylene again added to a final concentration of 10 ppm. After 48 hours, each plant was assessed for retention of flowers. This treatment normally induces flowers to fall off (abscise) by the end of the treatment. Plants are evaluated on the basis of flower retention with good lines maintaining the flowers. Typically, a normal, non-drop transgenic plant will drop >95% of its flowers by the end of treatment, indicating that it is an effective stress assay.

TABLE 2
Results of flower abscission tests.

| Line | # Plants | # abscissions | % retention | Significant |
|---|---|---|---|---|
| 67 | 10 | 6/44 | 86 | * |
| 68 | 7 | 23/39 | 41 | * |
| 96 | 40 | 122/205 | 40 | * |
| 133 | 19 | 69/104 | 36 | * |
| 178 | 5 | 20/23 | 13 | |
| 181 | 4 | 25/26 | 4 | |
| 184 | 4 | 19/19 | 0 | |
| 187 | 1 | 2/5 | 60 | * |
| 190 | 1 | 7/7 | 0 | |
| 194 | 5 | 15/23 | 30 | * |
| wild type | 12 | 48/50 | 4 | |
| 197 | 4 | 21/21 | 0 | |
| 198 | 4 | 24/27 | 11 | |
| 199 | 3 | 16/16 | 0 | |
| 200 | 4 | 22/24 | 8 | |
| 202 | 2 | 7/7 | 0 | |
| 203 | 4 | 18/20 | 10 | |
| 204 | 4 | 21/21 | 0 | |
| Plants of each independent transgenic line and controls (wild type) were placed in a sealed container. Ethylene gas was injected into the container to a final concentration of 10.0 parts per million. The number of flowers abscissed over the total number of flowers is indicated in Row 3. | | | | |
| # Note that very young flowers do not typically abscise with this treatment since abscission layer do not form until flowers are nearly mature. Those numbers considered to be substantially improved relative to wild type are indicated by * in the final Row. | | | | |

Of the 17 evaluated lines, six showed a high level of flower retention. These ranged from a low of 30% to a high of 86% flower retention. Importantly, all of the lines exhibited a normal pattern of fruit ripening. Since ripening is absolutely dependent upon ethylene action, ripening of the fruits indicates that the lines that retain flowers are not expressing a generalized ethylene insensitivity. Rather, they must have gained a significant degree of ethylene insensitivity in the target tissues. It is noteworthy that even a 30% retention rate under such an extreme stress would be highly desirable from a yield point of view.

It should be understood that the examples and embodiments described herein are for illustrative purposes only and that various modifications or changes in light thereof will be suggested to persons skilled in the art and are to be included within the spirit and purview of this application and the scope of the appended claims.

### REFERENCES

U.S. Pat. No. 6,294,716
U.S. Pat. No. 5,824,868
U.S. Pat. No. 5,689,055
U.S. Pat. No. 5,399,680
Published U.S. patent application 20010019995
Alonso, J. M., Hirayama, T., Roman, G., Nourizadeh, S., Ecker, J. R. (1999) “EIN2, a bifunctional transducer of ethylene and stress responses in Arabidopsis” Science 284(5423):2148-2152.
Altschul, S. F. et al. (1990) “Basic Local Alignment Search Tool” J. Mol. Biol. 215:403-410.
Altschul, S. F. et al. (1997) “Gapped BLAST and PSI-BLAST: A New Generation of Protein Database Search Programs” Nucl. Acids Res. 25:3389-3402.
Beltz, G. A., K. A. Jacobs, T. H. Eickbush, P. T. Cherbas, and F. C. Kafatos (1983) Methods of Enzymology, R. Wu, L. Grossman and K. Moldave [eds.] Academic Press, New York 100:266-285.
Chang, C., Kwok, S. F., Bleecker, A. B. Meyerowitz, E. M. (1993) “Arabidopsis ethylene response gene ETR1-Similarity of product to two-component regulators” Science 262:539-544.
S Chao, Q. M., Rothenberg, M., Solano, R., Roman, G., Terzaghi, W., Ecker, J. R. (1997) “Activation of the ethylene gas response pathway in Arabidopsis by the nuclear protein ETHYLENE-INSENSITIVE3 and related proteins” Cell 89(7):1133-1144.
Hua, J., Chang, C., Sun, Q., Meyerowitz, E. M. (1995) “Ethylene insensitivity conferred by Arabidopsis ERS gene” Science 269:1712-1714.
Hua, J., Meyerowitz, E. M. (1998a) “Ethylene responses are negatively regulated by a receptor gene family in Arabidopsis thaliana” Cell 94:261-271.
Hua, J., Sakai, H. Nourizadeh, S., Chen, Q. G., Bleecker, A. B., Ecker, J. R., Meyerowitz, E. M. (1998b) “EIN4 and ERS2 are members of the putative ethylene receptor gene family in Arabidopsis” Plant Cell 10:1321-1332.
Karlin, S., Altschul, S. F. (1990) “Methods for Assessing the Statistical Significance of Molecular Sequence Features by Using General Scoring Schemes” Proc. Natl. Acad. Sci. USA 87:2264-2268.
Karlin, S., Altschul, S. F. (1993) “Applications and Statistics for Multiple High-Scoring Segments in Molecular Sequences” Proc. Natl. Acad. Sci. USA 90:5873-5877.
Koehler, S. M., Matters, G. L., Nath, P., Kemmerer, E. C., Tucker, M. L. (1996) “The gene promoter for a bean abscission cellulase is ethylene-induced in transgenic tomato and shows high sequence conservation with a soybean abscission cellulase” Plant Molecular Biology 31:595-606.
Maniatis, T., E. F. Fritsch, J. Sambrook (1982) Molecular Cloning: A Laboratory Manual, Cold Spring Harbor Laboratory, Cold Spring Harbor, N.Y.
McCormick, S., Niedermeyer, J., Fry, J., Bamason, A., Horsch, R., Fraley, R. (1986) Plant Cell Reports 5:81-84.
Sakai, H., Hua, J., Chen, Q. G., Chang, C., Medrano, L. J., Bleecker, A. B., Meyerowitz, E. M. (1998) “ETR2 is an ETR1-like gene involved in ethylene signaling in Arabidopsis” Proc. Natl. Acad. Sci. USA 95:5812-5817.
Tieman, D., Ciardi, J., Taylor, M., Klee, H. (2001) “Members of the tomato LeEIL (EIN3-like) gene family are functionally redundant and regulate ethylene responses throughout plant development” Plant J 26:47-58.
Yang, T. T. et al. (1996) “Optimized Codon Usage and Chromophore Mutations Provide Enhanced Sensitivity with the Green Fluorescent Protein” Nucleic Acid Research 24(22):4592-4593.

We claim:

1. A polynucleotide comprising:
a) a nucleotide sequence encoding a mutant etr1 plant ethylene receptor protein, or a fragment thereof, wherein said mutant protein, or said fragment thereof, exhibits ethylene insensitivity; and
b) a regulatory nucleotide sequence operably linked to said protein encoding nucleotide sequence, wherein said regulatory nucleotide sequence promotes transcription of said protein encoding nucleotide sequence in cells that comprise abscission zone tissue of a plant, and wherein said regulatory nucleotide sequence comprises a promoter sequence from a cotton chitinase gene comprising the nucleotide sequence shown in SEQ ID NO: 8.

2. The polynucleotide according to claim 1, wherein said etr1 mutant receptor has a sequence of an Arabidopsis thaliana etr1 sequence.

3. The polynucleotide according to claim 2, wherein said mutant receptor sequence is the etr1-1 sequence (SEQ ID NO: 1).

4. The polynucleotide according to claim 2, wherein said mutant receptor sequence is the etr1-2 sequence (SEQ ID NO: 2).

5. The polynucleotide according to claim 2, wherein said mutant receptor sequence is the etr1-3 sequence (SEQ ID NO: 3).

6. The polynucleotide according to claim 2, wherein said mutant receptor sequence is the etr1-4 sequence (SEQ ID NO: 4).

7. The polynucleotide according to claim 3, wherein said nucleotide sequence encoding said mutant receptor sequence comprises the sequence shown in SEQ ID NO: 5.

8. A cell transformed with a polynucleotide that comprises:
a) a nucleotide sequence encoding a mutant etr1 plant ethylene receptor protein, or a fragment thereof, wherein said mutant protein, or said fragment thereof, exhibits ethylene insensitivity; and
b) a regulatory nucleotide sequence operably linked to said protein encoding nucleotide sequence, wherein said regulatory nucleotide sequence promotes transcription of said protein encoding nucleotide sequence in cells that comprise abscission zone tissue of a plant, and wherein said regulatory nucleotide sequence comprises a promoter sequence from a cotton chitinase gene comprising the nucleotide sequence shown in SEQ ID NO: 8.

9. A plant, plant tissue, or a plant cell transformed with or bred to contain a polynucleotide that comprises:
a) a nucleotide sequence encoding a mutant etr1 plant ethylene receptor protein, or a fragment thereof, wherein said mutant protein, or said fragment thereof, exhibits ethylene insensitivity; and
b) a regulatory nucleotide sequence operably linked to said protein encoding nucleotide sequence, wherein said regulatory nucleotide sequence promotes transcription of said protein encoding nucleotide sequence in cells that comprise abscission zone tissue of a plant, and wherein said regulatory nucleotide sequence comprises a promoter sequence from a cotton chitinase gene comprising the nucleotide sequence shown in SEQ ID NO: 8.

10. The plant, plant tissue, or a plant cell according to claim 9, wherein said etr1 mutant receptor has a sequence of an Arabidopsis thaliana etr1 sequence.

11. The plant, plant tissue, or a plant cell according to claim 10, wherein said mutant receptor sequence is the etr1-1 sequence (SEQ ID NO: 1).

12. The plant, plant tissue, or a plant cell according to claim 10, wherein said mutant receptor sequence is the etr1-2 sequence (SEQ ID NO: 2).

13. The plant, plant tissue, or a plant cell according to claim 10, wherein said mutant receptor sequence is the etr1-3 sequence (SEQ ID NO: 3).

14. The plant, plant tissue, or a plant cell according to claim 10, wherein said mutant receptor sequence is the etr1-4 sequence (SEQ ID NO: 4).

15. The plant, plant tissue, or a plant cell according to claim 11, wherein said nucleotide sequence encoding said mutant receptor sequence comprises the sequence shown in SEQ ID NO: 5.

16. The plant, plant tissue, or a plant cell according to claim 9, wherein said plant is a monocotyledonous plant.

17. The plant, plant tissue, or a plant cell according to claim 16, wherein said monocotyledonous plant is selected from the group consisting of rice, wheat, barley, oats, rye, sorghum, maize, lilies, banana, pineapple, turfgrass, gladiolus, and millet.

18. The plant, plant tissue, or a plant cell according to claim 9, wherein said plant is a dicotyledonous plant.

19. The plant, plant tissue, or a plant cell according to claim 18, wherein said dicotyledonous plant is selected from the group consisting of cotton, peas, alfalfa, chickpea, chicory, clover, kale, lentil, prairie grass, soybean, tobacco, potato, sweet potato, radish, cabbage, rape, apple trees, coffee, tomato, melon, citrus, beans, roses, sugar beet, squash, peppers, strawberry, carnation, chrysanthemums, impatiens, eucalyptus, and lettuce.

20. A method for decreasing flower, fruit, or leaf drop in a plant upon exposure to ethylene, said method comprising introducing a polynucleotide into said plant, wherein said polynucleotide comprises:
a) a nucleotide sequence encoding a mutant etr1 plant ethylene receptor protein, or a fragment thereof, wherein said mutant protein, or said fragment thereof, exhibits ethylene insensitivity; and
b) a regulatory nucleotide sequence operably linked to said protein encoding nucleotide sequence, wherein said regulatory nucleotide sequence promotes transcription of said protein encoding nucleotide sequence in cells that comprise abscission zone tissue of a plant, and wherein said regulatory nucleotide sequence comprises a promoter sequence from a cotton chitinase gene comprising the nucleotide sequence shown in SEQ ID NO: 8.

21. The method according to claim 20, wherein said etr1 mutant receptor has a sequence of an Arabidopsis thaliana etr1 sequence.

22. The method according to claim 21, wherein said mutant receptor sequence is the etr1-1 sequence (SEQ ID NO: 1).

23. The method according to claim 21, wherein said mutant receptor sequence is the etr1-2 sequence (SEQ ID NO: 2).

24. The method according to claim 21, wherein said mutant receptor sequence is the etr1-3 sequence (SEQ ID NO: 3).

25. The method according to claim 21, wherein said mutant receptor sequence is the etr1-4 sequence (SEQ ID NO: 4).

26. The method according to claim 22, wherein said nucleotide sequence encoding said mutant receptor sequence comprises the sequence shown in SEQ ID NO: 5.

27. The method according to claim 20, wherein said plant is a monocotyledonous plant.

28. The method according to claim 27, wherein said monocotyledonous plant is selected from the group consisting of rice, wheat, barley, oats, rye, sorghum, maize, lilies, banana, pineapple, turfgrass, gladiolus, and millet.

29. The method according to claim 20, wherein said plant is a dicotyledonous plant.

30. The method according to claim 29, wherein said dicotyledonous plant is selected from the group consisting of cotton, peas, alfalfa, chickpea, chicory, clover, kale, lentil, prairie grass, soybean, tobacco, potato, sweet potato, radish, cabbage, rape, apple trees, coffee, tomato, melon, citrus, beans, roses, sugar beet, squash, peppers, strawberry, carnation, chrysanthemums, impatiens, eucalyptus, and lettuce.


SEQUENCE LISTING (16 sequences)

SEQ ID NO: 1 - protein, 738 aa, Arabidopsis thaliana
MEVCNCIEPQWPADELLMKYQYISDFFIAIAYFSIPLELIYFVKKSAVFPYRWVLVQFGAFIVLYGATHLINLWTFTTHSRTVALVMTTAKVLTAVVSCATALMLVHIIPDLLSVKTRELFLKNKAAELDREMGLIRTQEETGRHVRMLTHEIRSTLDRHTILKTTLVELGRTLALEECALWMPTRTGLELQLSYTLRHQHPVEYTVPIQLPVINQVFGTSRAVKISPNSPVARLRPVSGKYMLGEVVAVRVPLLHLSNFQINDWPELSTKRYALMVLMLPSDSARQWHVHELELVEVVADQVAVALSHAAILEESMRARDLLMEQNVALDLARREAETAIRARNDFLAVMNHEMRTPMHAIIALSSLLQETELTPEQRLMVETILKSSNLLATLMNDVLDLSRLEDGSLQLELGTFNLHTLFREVLNLIKPIAVVKKLPITLNLAPDLPEFVVGDEKRLMQIILNIVGNAVKFSKQGSISVTALVTKSDTRAADFFVVPTGSHFYLRVKVKDSGAGINPQDIPKIFTKFAQTQSLATRSSGGSGLGLAISKRFVNLMEGNIWIESDGLGKGCTAIFDVKLGISERSNESKQSGIPKVPAIPRHSNFTGLKVLVMDENGVSRMVTKGLLVHLGCEVTTVSSNEECLRVVSHEHKVVFMDVCMPGVENYQIALRIHEKFTKQRHQRPLLVALSGNTDKSTKEKCMSFGLDGVLLKPVSLDNIRDVLSDLLEPRVLYEGM

SEQ ID NO: 2 - protein, 738 aa, Arabidopsis thaliana
MEVCNCIEPQWPADELLMKYQYISDFFIAIAYFSIPLELIYFVKKSAVFPYRWVLVQFGAFIVLYGATHLINLWTFTTHSRTVALVMTTAKVLTAVVSCATTLMLVHIIPDLLSVKTRELFLKNKAAELDREMGLIRTQEETGRHVRMLTHEIRSTLDRHTILKTTLVELGRTLALEECALWMPTRTGLELQLSYTLRHQHPVEYTVPIQLPVINQVFGTSRAVKISPNSPVARLRPVSGKYMLGEVVAVRVPLLHLSNFQINDWPELSTKRYALMVLMLPSDSARQWHVHELELVEVVADQVAVALSHAAILEESMRARDLLMEQNVALDLARREAETAIRARNDFLAVMNHEMRTPMHAIIALSSLLQETELTPEQRLMVETILKSSNLLATLMNDVLDLSRLEDGSLQLELGTFNLHTLFREVLNLIKPIAVVKKLPITLNLAPDLPEFVVGDEKRLMQIILNIVGNAVKFSKQGSISVTALVTKSDTRAADFFVVPTGSHFYLRVKVKDSGAGINPQDIPKIFTKFAQTQSLATRSSGGSGLGLAISKRFVNLMEGNIWIESDGLGKGCTAIFDVKLGISERSNESKQSGIPKVPAIPRHSNFTGLKVLVMDENGVSRMVTKGLLVHLGCEVTTVSSNEECLRVVSHEHKVVFMDVCMPGVENYQIALRIHEKFTKQRHQRPLLVALSGNTDKSTKEKCMSFGLDGVLLKPVSLDNIRDVLSDLLEPRVLYEGM

SEQ ID NO: 3 - protein, 738 aa, Arabidopsis thaliana
MEVCNCIEPQWPADELLMKYQYISDFFIAIVYFSIPLELIYFVKKSAVFPYRWVLVQFGAFIVLYGATHLINLWTFTTHSRTVALVMTTAKVLTAVVSCATALMLVHIIPDLLSVKTRELFLKNKAAELDREMGLIRTQEETGRHVRMLTHEIRSTLDRHTILKTTLVELGRTLALEECALWMPTRTGLELQLSYTLRHQHPVEYTVPIQLPVINQVFGTSRAVKISPNSPVARLRPVSGKYMLGEVVAVRVPLLHLSNFQINDWPELSTKRYALMVLMLPSDSARQWHVHELELVEVVADQVAVALSHAAILEESMRARDLLMEQNVALDLARREAETAIRARNDFLAVMNHEMRTPMHAIIALSSLLQETELTPEQRLMVETILKSSNLLATLMNDVLDLSRLEDGSLQLELGTFNLHTLFREVLNLIKPIAVVKKLPITLNLAPDLPEFVVGDEKRLMQIILNIVGNAVKFSKQGSISVTALVTKSDTRAADFFVVPTGSHFYLRVKVKDSGAGINPQDIPKIFTKFAQTQSLATRSSGGSGLGLAISKRFVNLMEGNIWIESDGLGKGCTAIFDVKLGISERSNESKQSGIPKVPAIPRHSNFTGLKVLVMDENGVSRMVTKGLLVHLGCEVTTVSSNEECLRVVSHEHKVVFMDVCMPGVENYQIALRIHEKFTKQRHQRPLLVALSGNTDKSTKEKCMSFGLDGVLLKPVSLDNIRDVLSDLLEPRVLYEGM

SEQ ID NO: 4 - protein, 738 aa, Arabidopsis thaliana
MEVCNCIEPQWPADELLMKYQYISDFFIAIAYFSIPLELIYFVKKSAVFPYRWVLVQFGAFFVLYGATHLINLWTFTTHSRTVALVMTTAKVLTAVVSCATALMLVHIIPDLLSVKTRELFLKNKAAELDREMGLIRTQEETGRHVRMLTHEIRSTLDRHTILKTTLVELGRTLALEECALWMPTRTGLELQLSYTLRHQHPVEYTVPIQLPVINQVFGTSRAVKISPNSPVARLRPVSGKYMLGEVVAVRVPLLHLSNFQINDWPELSTKRYALMVLMLPSDSARQWHVHELELVEVVADQVAVALSHAAILEESMRARDLLMEQNVALDLARREAETAIRARNDFLAVMNHEMRTPMHAIIALSSLLQETELTPEQRLMVETILKSSNLLATLMNDVLDLSRLEDGSLQLELGTFNLHTLFREVLNLIKPIAVVKKLPITLNLAPDLPEFVVGDEKRLMQIILNIVGNAVKFSKQGSISVTALVTKSDTRAADFFVVPTGSHFYLRVKVKDSGAGINPQDIPKIFTKFAQTQSLATRSSGGSGLGLAISKRFVNLMEGNIWIESDGLGKGCTAIFDVKLGISERSNESKQSGIPKVPAIPRHSNFTGLKVLVMDENGVSRMVTKGLLVHLGCEVTTVSSNEECLRVVSHEHKVVFMDVCMPGVENYQIALRIHEKFTKQRHQRPLLVALSGNTDKSTKEKCMSFGLDGVLLKPVSLDNIRDVLSDLLEPRVLYEGM

SEQ ID NO: 5 - DNA, 2214 bp, Arabidopsis thaliana
  CDS (1)..(2214)
atggaagtctgcaattgtattgaaccgcaatggccagcggatgaattgttaatgaaataccaatacatctccgatttcttcattgcgattgcgtatttttcgattcctcttgagttgatttactttgtgaagaaatcagccgtgtttccgtatagatgggtacttgttcagtttggtgcttttatcgttctttatggagcaactcatcttattaacttatggactttcactacgcattcgagaaccgtggcgcttgtgatgactaccgcgaaggtgttaaccgctgttgtctcgtgtgctactgcgttgatgcttgttcatattattcctgatcttttgagtgttaagactcgggagcttttcttgaaaaataaagctgctgagctcgatagagaaatgggattgattcgaactcaggaagaaaccggaaggcatgtgagaatgttgactcatgagattagaagcactttagatagacatactattttaaagactacacttgttgagcttggtaggacattagctttggaggagtgtgcattgtggatgcctactagaactgggttagagctacagctttcttatacacttcgtcatcaacatcccgtggagtatacggttcctattcaattaccggtgattaaccaagtgtttggtactagtagggctgtaaaaatatctcctaattctcctgtggctaggttgagacctgtttctgggaaatatatgctaggggaggtggtcgctgtgagggttccgcttctccacctttctaattttcagattaatgactggcctgagctttcaacaaagagatatgctttgatggttttgatgcttccttcagatagtgcaaggcaatggcatgtccatgagttggaactcgttgaagtcgtcgctgatcaggtggctgtagctctctcacatgctgcgatcctagaagagtcgatgcgagctagggaccttctcatggagcagaatgttgctcttgatctagctagacgagaagcagaaacagcaatccgtgcccgcaatgatttcctagcggttatgaaccatgaaatgcgaacaccgatgcatgcgattattgcactctcttccttactccaagaaacggaactaacccctgaacaaagactgatggtggaaacaatacttaaaagtagtaaccttttggcaactttgatgaatgatgtcttagatctttcaaggttagaagatggaagtcttcaacttgaacttgggacattcaatcttcatacattatttagagaggtcctcaatctgataaagcctatagcggttgttaagaaattacccatcacactaaatcttgcaccagatttgccagaatttgttgttggggatgagaaacggctaatgcagataatattaaatatagttggtaatgctgtgaaattctccaaacaaggtagtatctccgtaaccgctcttgtcaccaagtcagacacacgagctgctgacttttttgtcgtgccaactgggagtcatttctacttgagagtgaaggtaaaagactctggagcaggaataaatcctcaagacattccaaagattttcactaaatttgctcaaacacaatctttagcgacgagaagctcgggtggtagtgggcttggcctcgccatctccaagaggtttgtgaatctgatggagggtaacatttggattgagagcgatggtcttggaaaaggatgcacggctatctttgatgttaaacttgggatctcagaacgttcaaacgaatctaaacagtcgggcataccgaaagttccagccattccccgacattcaaatttcactggacttaaggttcttgtcatggatgagaacggggtaagtagaatggtgacgaagggacttcttgtacaccttgggtgcgaagtgaccacggtgagttcaaacgaggagtgtctccgagttgtgtcccatgagcacaaagtggtcttcatggacgtgtgcatgcccggggtcgaaaactaccaaatcgctctccgtattcacgagaaattcacaaaacaacgccaccaacggccactacttgtggcactcagtggtaacactgacaaatccacaaaagagaaatgcatgagctttggtctagacggtgtgttgctcaaacccgtatcactagacaacataagagatgttctgtctgatcttctcgagccccgggtactgtacgagggcatg

SEQ ID NO: 6 - protein, 773 aa, Arabidopsis thaliana
MVKEIASWLLILSMVVFVSPVLAINGGGYPRCNCEDEGNSFWSTENILETQRVSDFLIAVAYFSIPIELLYFVSCSNVPFKWVLFEFIAFIVLCGMTHLLHGWTYSAHPFRLMMAFTVFKMLTALVSCATAITLITLIPLLLKVKVREFMLKKKAHELGREVGLILIKKETGFHVRMLTQEIRKSLDRHTILYTTLVELSKTLGLQNCAVWMPNDGGTEMDLTHELRGRGGYGGCSVSMEDLDVVRIRESDEVNVLSVDSSIARASGGGGDVSEIGAVAAIRMPMLRVSDFNGELSYAILVCVLPGGTPRDWTYQEIEIVKVVADQVTVALDHAAVLEESQLMREKLAEQNRALQMAKRDALRASQARNAFQKTMSEGMRRPMHSILGLLSMIQDEKLSDEQKMIVDTMVKTGNVMSNLVGDSMDVPDGRFGTEMKPFSLHRTIHEAACMARCLCLCNGIRFLVDAEKSLPDNVVGDERRVFQVILHIVGSLVKPRKRQEGSSLMFKVLKERGSLDRSDHRWAAWRSPASSADGDVYIRFEMNVENDDSSSQSFASVSSRDQEVGDVRFSGGYGLGQDLSFGVCKKVVQLIHGNISVVPGSDGSPETMSLLLRFRRRPSISVHGSSESPAPDHHAHPHSNSLLRGLQVLLVDTNDSNRAVTRKLLEKLGCDVTAVSSGFDCLTAIAPGSSSPSTSFQVVVLDLQMAEMDGYEVAMRIRSRSWPLIVATTVSLDEEMWDKCAQIGINGVVRKPVVLRAMESELRRVLLQADQLL

SEQ ID NO: 7 - DNA, 2893 bp, Arabidopsis thaliana
  CDS (397)..(2715)
atacatacacttctctttttatcctccatcggcggcttatggcggttttccggcactaatcatctccggcatatataaataaacgtacttcacgtttttttatataacttcaaagtagtttcagatttgtctctatctcttcacttttaagtcttctggttttgtcatcaccagctttttttgttctgtctctgtctttctctttgtgtatttttattctcgtcatcgttgttcttctatgagaggaagatcggaatgtcgaagagaattagaagattctcgtacatcacttcgttggaatttcacaggtcgatgagagatctgagaactgtttcattttgatccaaactcatctctttcagagtataatggactaagcattttttttctccgaagatggttaaagaaatagcttcttggttattgatactatcaatggtggtgtttgtttctccggttttagctataaacggcggtggttatccacgatgtaactgcgaagacgaaggaaacagtttctggagtacagagaacattctagaaactcaaagagtaagcgatttcttaatcgcagtagcttatttctcaatccctattgagttactttacttcgtgagttgttccaatgttccattcaaatgggttctctttgagtttatcgccttcattgttctttgtggtatgactcatcttcttcatggttggacttactctgctcatccatttagattaatgatggcgtttactgttttcaagatgttgactgctttagtctcttgtgctactgcgattacgcttattactttgattcctctgcttttgaaagttaaagttagagagtttatgcttaagaagaaagctcatgagcttggtcgtgaagttggtttgattttgattaagaaagagactggctttcatgttcgtatgcttactcaagagattcgtaagtctttggatcgtcatacgattctttatactactttggttgagctttcgaagactttagggttgcagaattgtgcggtttggatgccgaatgacggtggaacggagatggatttgactcatgagttgagagggagaggtggttatggtggttgttctgtttctatggaggatttggatgttgttaggattagggagagtgatgaagtgaatgtgttgagtgttgactcgtccattgctcgagctagtggtggtggtggggatgttagtgagattggtgccgtggctgctattagaatgccgatgcttcgtgtttcggattttaatggagagctaagttatgcgatacttgtttgtgttttaccgggcgggacgcctcgggattggacttatcaggagattgagattgttaaagttgtggcggatcaagtaaccgttgcgttagatcatgcagcggttcttgaagagtctcagcttatgagggagaagctggcggaacagaacagggcgttgcagatggcgaagagagacgcgttgagagcgagccaagcgaggaatgcgtttcagaaaacgatgagcgaagggatgaggcgtcctatgcattcgatactcggtcttttgtcgatgattcaggacgagaagttgagtgacgagcagaaaatgattgttgatacgatggttaaaacagggaatgttatgtcgaatttggtgggggactctatggatgtgcctgacggtagatttggtacggagatgaaaccatttagtctgcatcgtacgatccatgaagcagcttgtatggcgagatgtttgtgtctatgcaatggaattaggttcttggttgacgcggagaagtctctacctgataatgtagtaggtgatgaaagaagggtctttcaagtgatacttcatatagttggtagtttagtaaagcctagaaaacgtcaagaaggatcttcattgatgtttaaggttttgaaagaaagaggaagcttggataggagtgatcatagatgggctgcttggagatcaccggcttcttcagcagatggagatgtgtatataagatttgaaatgaatgtagagaatgatgattcaagttctcaatcatttgcttctgtttcctccagagatcaagaagttggtgatgttagattctccggcggctatgggttaggacaagatctaagctttggtgtttgtaagaaagtggtgcagttgattcatgggaatatctcggtggtccctggctcggatggttcaccggagaccatgtcgttgctccttcggtttcgacgtagaccctccatatctgtccatggatccagcgagtcgccagctcctgaccaccacgctcacccacattcgaattctctgttacgtggcttacaagttttattggtagacaccaacgattcgaaccgggcagttacacgtaaactcttagagaaactcgggtgcgatgtaaccgcggtttcctctggattcgattgccttaccgccattgctcccggctcgtcctcgccttctacttcgtttcaagtggtggtgcttgatcttcaaatggcagagatggacggttatgaagtggccatgaggatcaggagtcgatcttggccgttgattgtggcgacgacagtgagcttggatgaagaaatgtgggacaagtgtgcacagattggaatcaatggagttgtgagaaagccagtggtgttaagagctatggagagtgagctccgaagagtattgttgcaagctgaccaacttctctaagttgttatctcaacttctcttctacattcaaaatttttacaccatagatttatgtcaaatatatcaaaatgaaatttcgaaattgttattatatataccacccatatctctatgatttgtacatcctgtttttttttgttctttttctcattttgaaccccacgaaattgcattg

SEQ ID NO: 8 - DNA, 1622 bp, Gossypium sp.
agcttgcggccgctatttagggcctttttagatagatgatgcgtttatctacaattagtataaaattagctttggtggtgaaattaaccattatagcgtgaaacaaaaagaaaagtaaatatcactggaggtaaacaaaaagaaaggtaaagaaaggctcctaatcagatgaaaatttctctcaaacttttagcactcttttggaacgtaaacagtgaagtgaactagagatgttaaaagaggaagataaatggttaaatatacaaaatgtgtttaacataaatccatttatcccccattaattcaatcctttgaattgttaaatatataatatatgtttaatgtcttcctgttttaaccatggtaaaggagtcttcttgatttgttaattaagtgaaaggcaccaaaaccaaatttatagcgtttaaattgagtacattttgtatatacggtcgaaagccgcctcagggttcaatggctgctaatatttgcccaaaataaaatctcctttaggacttagcttcgtgtacgatagccaattcgatatcttatgtaggtaactatgtttcgtaatacatagaattgttcaactttttttatgttcgaacttttttttaatttgcaatgtaattatttgtcttatgttacattttagtcatttatgtttaaaatgttatattttagttatttacgttatcgtgttgtaacattttaggtactgagccattaattgtcgttaacagtgtaacagaaagctgacgtggcacgttaaatcatcgcttcaaattaaaattttggattaaattatacaattggtccctatatttttttcatttttttctttatttttcattctcttcggcttctccctatgttttcctctcttcttcatttcttttaacatagtttttttatattttctaaaattaaatttttcaaaaaaaataaaatataggagttagttttaaaaaatacgttaaaagaaatggataagggaggaaaacagagggaaaagcagaagaggatgaaagaaaaagaaagttaaaagaacataaaagaaaaaattaaattgctctaaaagaaaaaatatggggacagattgtataaattagccaaaaatttttgtttaaaatgatgatttaagtgctatgtcagtttaccattaaaggtaacaattaacgttttaataattaaaatattataacccgataaaataagtaactaaaattttaaattttaaatataaataactaaaatataacttgagataaataaaagttgctattaaaatttaggtatatcaacattaataatgttggccccatataagaagaattcatgtgcaagaagttaaaatgggtcaacagccgcctacatgccaacccttaatttccaacttttagttggtgaggataagatgagactaaaatccgacattgcatattgaaaggtcaagcataatcagataaaaaatcttggttcattcattgagtaccaagtaagtcacccttttttaatcaaaactacaacagtgaaggaaactacgaataatctatcattcaccatgactttctcccaccatatatttatgtacacccttccaatcttcctacactaccaca

SEQ ID NO: 9 - DNA, 11606 bp, Artificial sequence
  plasmid construct encoding mutant ethylene receptor
  CDS (1674)..(3890)
agcttgcggccgctatttagggcctttttagatagatgatgcgtttatctacaattagtataaaattagctttggtggtgaaattaaccattatagcgtgaaacaaaaagaaaagtaaatatcactggaggtaaacaaaaagaaaggtaaagaaaggctcctaatcagatgaaaatttctctcaaacttttagcactcttttggaacgtaaacagtgaagtgaactagagatgttaaaagaggaagataaatggttaaatatacaaaatgtgtttaacataaatccatttatcccccattaattcaatcctttgaattgttaaatatataatatatgtttaatgtcttcctgttttaaccatggtaaaggagtcttcttgatttgttaattaagtgaaaggcaccaaaaccaaatttatagcgtttaaattgagtacattttgtatatacggtcgaaagccgcctcagggttcaatggctgctaatatttgcccaaaataaaatctcctttaggacttagcttcgtgtacgatagccaattcgatatcttatgtaggtaactatgtttcgtaatacatagaattgttcaactttttttatgttcgaacttttttttaatttgcaatgtaattatttgtcttatgttacattttagtcatttatgtttaaaatgttatattttagttatttacgttatcgtgttgtaacattttaggtactgagccattaattgtcgttaacagtgtaacagaaagctgacgtggcacgttaaatcatcgcttcaaattaaaattttggattaaattatacaattggtccctatatttttttcatttttttctttatttttcattctcttcggcttctccctatgttttcctctcttcttcatttcttttaacatagtttttttatattttctaaaattaaatttttcaaaaaaaataaaatataggagttagttttaaaaaatacgttaaaagaaatggataagggaggaaaacagagggaaaagcagaagaggatgaaagaaaaagaaagttaaaagaacataaaagaaaaaattaaattgctctaaaagaaaaaatatggggacagattgtataaattagccaaaaatttttgtttaaaatgatgatttaagtgctatgtcagtttaccattaaaggtaacaattaacgttttaataattaaaatattataacccgataaaataagtaactaaaattttaaattttaaatataaataactaaaatataacttgagataaataaaagttgctattaaaatttaggtatatcaacattaataatgttggccccatataagaagaattcatgtgcaagaagttaaaatgggtcaacagccgcctacatgccaacccttaatttccaacttttagttggtgaggataagatgagactaaaatccgacattgcatattgaaaggtcaagcataatcagataaaaaatcttggttcattcattgagtaccaagtaagtcacccttttttaatcaaaactacaacagtgaaggaaactacgaataatctatcattcaccatgactttctcccaccatatatttatgtacacccttccaatcttcctacactaccacatcgataaataaaaactgcagcccgggggatccatagtgtaaaaaattcataatggaagtctgcaattgtattgaaccgcaatggccagcggatgaattgttaatgaaataccaatacatctccgatttcttcattgcgattgcgtatttttcgattcctcttgagttgatttactttgtgaagaaatcagccgtgtttccgtatagatgggtacttgttcagtttggtgcttttatcgttctttatggagcaactcatcttattaacttatggactttcactacgcattcgagaaccgtggcgcttgtgatgactaccgcgaaggtgttaaccgctgttgtctcgtgtgctactgcgttgatgcttgttcatattattcctgatcttttgagtgttaagactcgggagcttttcttgaaaaataaagctgctgagctcgatagagaaatgggattgattcgaactcaggaagaaaccggaaggcatgtgagaatgttgactcatgagattagaagcactttagatagacatactattttaaagactacacttgttgagcttggtaggacattagctttggaggagtgtgcattgtggatgcctactagaactgggttagagctacagctttcttatacacttcgtcatcaacatcccgtggagtatacggttcctattcaattaccggtgattaaccaagtgtttggtactagtagggctgtaaaaatatctcctaattctcctgtggctaggttgagacctgtttctgggaaatatatgctaggggaggtggtcgctgtgagggttccgcttctccacctttctaattttcagattaatgactggcctgagctttcaacaaagagatatgctttgatggttttgatgcttccttcagatagtgcaaggcaatggcatgtccatgagttggaactcgttgaagtcgtcgctgatcaggtggctgtagctctctcacatgctgcgatcctagaagagtcgatgcgagctagggaccttctcatggagcagaatgttgctcttgatctagctagacgagaagcagaaacagcaatccgtgcccgcaatgatttcctagcggttatgaaccatgaaatgcgaacaccgatgcatgcgattattgcactctcttccttactccaagaaacggaactaacccctgaacaaagactgatggtggaaacaatacttaaaagtagtaaccttttggcaactttgatgaatgatgtcttagatctttcaaggttagaagatggaagtcttcaacttgaacttgggacattcaatcttcatacattatttagagaggtcctcaatctgataaagcctatagcggttgttaagaaattacccatcacactaaatcttgcaccagatttgccagaatttgttgttggggatgagaaacggctaatgcagataatattaaatatagttggtaatgctgtgaaattctccaaacaaggtagtatctccgtaaccgctcttgtcaccaagtcagacacacgagctgctgacttttttgtcgtgccaactgggagtcatttctacttgagagtgaaggtaaaagactctggagcaggaataaatcctcaagacattccaaagattttcactaaatttgctcaaacacaatctttagcgacgagaagctcgggtggtagtgggcttggcctcgccatctccaagaggtttgtgaatctgatggagggtaacatttggattgagagcgatggtcttggaaaaggatgcacggctatctttgatgttaaacttgggatctcagaacgttcaaacgaatctaaacagtcgggcataccgaaagttccagccattccccgacattcaaatttcactggacttaaggttcttgtcatggatgagaacggggtaagtagaatggtgacgaagggacttcttgtacaccttgggtgcgaagtgaccacggtgagttcaaacgaggagtgtctccgagttgtgtcccatgagcacaaagtggtcttcatggacgtgtgcatgcccggggtcgaaaactaccaaatcgctctccgtattcacgagaaattcacaaaacaacgccaccaacggccactacttgtggcactcagtggtaacactgacaaatccacaaaagagaaatgcatgagctttggtctagacggtgtgttgctcaaacccgtatcactagacaacataagagatgttctgtctgatcttctcgagccccgggtactgtacgagggcatgtaaggatccagctttcgttcgtatcatcggtttcgacaacgttcgtcaagttcaatgcatcagtttcattgcgcacacaccagaatcctactgagttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgcagccactcgaagcggccgccactcgagtggaagctagcttcccgatcctatctgtcacttcatcaaaaggacagtagaaaaggaaggtggcactacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatacttccactgacgtaagggatgacgcacaatcccactatccttcgcaagacccttcctctatataaggaagttcatttcatttggagaggacacgctgaaatcaccagtctctctctacaagatcggggatctctagctagacgatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgatccccaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatcgggccactcgagtggtggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttcctagcttggctgccatttttggggtgaggccgttcgcgcggggcgccagctggggggatgggaggcccgcgttaccgggagggttcgagaagggggggcaccccccttcggcgtgcgcggtcacgcgccagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcatcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgtggtatccacaacgccggcggccggccgcggtgtctcgcacacggcttcgacggcgtttctggcgcgtttgcagggccatagacggccgccagcccagcggcgagggcaaccagcccggtgagcgtcggaaagggtcgatcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggtcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagatgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgaggatttttcggcgctgcgctacgtccgckaccgcgttgagggatcaagccacagcagcccactcgacctctagccgacccagacgagccaagggatctttttggaatgctgctccgtcgtcaggctttccgacgtttgggtggttgaacagaagtcattatcgtacggaatgccaagcactcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccccatca

SEQ ID NO: 10 - DNA, 2458 bp, Lycopersicon esculentum
ggcacgagctgagttccagttgaaccacagttttgcaacgacataaaaaaagtgatctttttgaggctcatgtaatttgatcaatttcaattagattctgatctgaatattggattttgggtattagctgcttaatcctgtggaagttgcaagtagcttcacctttttcatcctgtggaagatgatgatgtttgaggaaatggggttctgtggggatcttgatttcttccctgctccgctgaaggaagtggaagtgtctgctccgcagagtcagactgagccggattctgtggtggatgatgattatagtgatgaggaggagattgaagtggatgagctggagaggaggatgtggagggacaagatgaagttgaaaaggctgaaagaaatgagtaaaagtaaggaaggtgttgatcctgcaaaacaacgtcagtcgcaggagcaggcgaggaggaagaagatgtcaagggcacaggatgggatcttgaagtacatgctgaaaatgatggaagtatgtaaagctcaggggtttgtttatgggatcattccggaaaaaggcaagccagttagtggggcatctgataatcttagggagtggtggaaggataaggtgaggttcgatcgaaatggtcctgcagcaatagccaaataccaagctgatcatgccatccctggcatgaacgaggggtctaatccagttggtcctacccctcacaccttgcaggagctgcaagataccacccttggttcgttattatcagctttgatgcagcactgtgatcctcctcagagaagatttccattggagaaaggtgttccaccaccatggtggcccacaggaaaggaggattggtggcctcaattgggtttgcagaaggaccaaggttctctaccttacaagaagcctcatgatctgaagaaggcgtggaaggttggtgtcctaacagcggtgatcaagcacatgttccctgatattgctaaaatccgcaagctggtaaggcagtcaaagtgcttacaggacaagatgacagccaaggaaagtgcaacttggcttgccatcatcagtcaggaggaagctttggctcgagaactctatcccgatcgctgtccacctttgtcctcagctggtgttagtggaaatttcatgttgaacgacagcagtgagtatgatgttgaaggtgctcaagatgagcctaactttgatgttcatgagcaaaaaccaaaccatctcaatctgttgaacatcagtgctgagagattcaaggagacgatgcctcttcagcaacaatctcatccaaacaaggatgaactggtcacaaacttagacttcagtctgaagaggaagcaagctaatgaacctactgtgatgatggatcaaaagatatacacatgcgagtttcttcaatgccctcacaatgaacttcgccatggttttcaggacagatcttccagagacaatcatcaatttgcttgcctttaccgaagttctacctgttttggagtttcaaactttcagattaatgaagtcaagccagttgtgttccctcaacaatatgtccagccaaagtcatctgctctgcctgttaatcaaggtccaccttcctttgatctatctggtataggagttcctgaagatgggcaaaggatgattaatgagcttatgtcaatctatgatagtgatgtacaaggaagcaaaaggcaaaatagggggaacattgcattgaccaaagagcagcctcatcaacaacctcgtgtccaccaggacaactacctgctcagccaagggataatggacggaaatatcttcaaaaacactaatatttccacaactcagtctatgctcccacaagtcgatccatttgatcaatccaaggctttcaatgcaggctccaacgacaacttccatttcatgtttgggtctccattcaacatacaatctaccaattataacggaaatctacctagcattggatacgataccacaccaaaacaagatgctcctatttggtactagccagccagaagtctgttgacatgttgtatatcaagtagtcagttttggcttcatagatatagcgatttctcatgggtaaaaagatcccatgtactcgtctgcttttttcattctggtggatttttcatcgcctatagtcctcttagttatggtgttttcagttaccatgtgttgtaaaggttgaagaactaggcatggtgtaaacaaacacccctcaaatacttgccaatgcttctcataggacatgtagatataaataagttacttgtttcatcatggccagttttatcagtctctttaaaactgttgaatttataggctatggttaaggtctttactccttgtttaatacttcttctcaagattgtttgggatatcaatgttgttattgttgccatatcttatatgatgtttttctttcattaaaaaaaaaaaaaaaaaaaa

SEQ ID NO: 11 - DNA, 2509 bp, Lycopersicon esculentum
ggcacgagtctaccttctatattctgaagggtcaattcttgaacaaaattttggaagcaaaaagaagaaacccctttgaggttgctggtttttttcaaaccaaaatttggctgccaagatgatgatgtttgaagacattgggttttgtgctgatcttgatttcttccctgctccgctgaaggaggcggaaacagtagctgctgttccaccaattgtgccggagccgatgatggatgatgatgatagtgatgaggagatcgatgtggatgagctggagaagaggatgtggagggataagatgaagctgaaaaggctgaaagaaatgagcaagggcaaggaaggtgttgatgctgtcaaacaacgccagtctcaggagcaagctaggaggaagaagatgtccagggctcaagatgggatcttgaagtacatgttgaagatgatggaagtctgtaaggctcagggttttgtttatggaattatcccggagaaaggcaaaccggtgactggggcatcggataatctcagggagtggtggaaggataaagtgaggtttgatcgcaatggacctgctgcgatagcaaagtaccaagctgacaatgccatccctggcaagaacgagggtgctaatccgattggtccaacccctcataccttgcaggagcttcaagataccacccttggttctctactgtcagctttaatgcaacattgtgatcctcctcagaggcgatttccattggaaaaaggtgtatcacctccatggtggccaaatggacaggaggattggtggcctcagttgggactgccaaatgatcaaggtcctccaccttacaagaagcctcatgatctgaagaaggcttggaaggttggtgtcctcacagcggtgatcaagcacatctcccctgatattgctaagatacgcaagctggtaaggcaatcgaagtgcttgcaggataagatgacagcaaaggaaagtgcaacttggcttgccatcatcaatcaggaggaagttttggctcgcgaactttatcctgatcgctgtccacctttgtcctcaggtggtagtagtggaaccttcactatgaacgacagcagtgagtatgatgttgaaggtgctattgatgaccctatctttgatgttcaagagcaaaaaccaaaccatctcagtttgctgaatgtcaatgttgagatgttcaaggagaagctgcctctgctacagcagtctcagccaatgaagggtgacatttttgccaacttagatttcactcgcaagaggaagccggctgatgacttgactttcctgatggatccgaagacatatacttgcgagtgtcttcattgccctcatagtgagcttcgcaatggttttccagacagatccagcagagacaatcatcagctaacttgcctcttcaggaatacttctcaatttgtagttccaaactttcacatggaggaggtcaagccagttgtcttccctcaacagtatgctgagccaaagcgggcttcgcttccggtcaacccagctccaccctcctttgatacatctggacttggggttcctgcagatgggcagagggtgatcaatgagcttatgtcattctatgaaagtaatgtgcaaggaaacaaaagttcaatggcggggaactctgtgatgtccaaagagcagcctcttcaacaacctagcattcaacagaacaattaccttcaaagccaagggaatgtgttggagggaagcatctttggggacaccaacatttctgctaacaactccatgtttgtgcagggtgatcggtttgatcagagcaaggttttaacttcaccattcaatgcaagctctactgatgatttcaatttcatgtttggatctccattcaacatgcaatccactgatctctctgaatgtctttctgggatttcacatgatgacgtgacgaagcaagatgcctcggtttggtactagcaatgcgattactcaaatgaacatcaagcagtggatatcacaatatacctttaatcaggtggatttcagtttgcctatagttctcagtttggtttttctgttaccatgtagttgtagaggttggaggatcgtgcatgacgttattacaagggagtagtaagagatgtcatgctgctggttagaaggaacttatagatgtttctaaataaggtttagcagtcagtttattttcatatatgtctataactctatataggttttagctttatcccatagtcaatcgttggtttcggagatatttaaacgcttttgtccctatatctggttctggaaatgttacaaattacctgcaggtgcagctgcaggtctttttgttatatgttatagtactatttgtcatggtggtggtgtaatatatacatatgtgttggccaaagctgtgtgatatgttgttagcatcctatataaggccatggcttgacaataaaatgttatgatgtattttggtatgtcctcttttcaccataaaaaaaaaaaaaaaaaaaa

SEQ ID NO: 12 - DNA, 2207 bp, Lycopersicon esculentum
ggcacgagcctctcttcttcttactttccaatacacaacaaaagtgaaaacatcacttcaatacacctaacattcttctcaaaccccttctctttctctcttttttcttttttttttggttcaaaatcaaagtagtatagccatagataacagcttcaacaagctgttttcaagaaaatcataatttggtaaatggggatatttgaagatatggggttctctggaaattttgagtttctatctgattctatgggatgtggagctcaagaagttgagcataagccggttgggttggaggaggatgattatagtgatgaggagatggatgtggaagagctagagaggaggatgtggagggatcgaatgcttttgaggcgtctcaaagagaaaaacaagaataaagtggtgggggatggtgcgaagcagcgtcagtcgcaggagcaggctcgtagaaagaagatgtcgcgtgcacaagatggtatactgaagtacatgctgaaaatgatggaggtttgtaatgctcagggttttgtttatggaattatccctgagaaagggaagcctgtgactggtgcttcggacaatcttcgtgcttggtggaaggaaaaggtcagatttgatcgaaatggccctgctgctattgctaagtatcaggctgataatcagattcctgggagagttgaggaatcgagtgtgatagtttccactccccacactttacaggagctgcaggatacaactctaggatcccttttgtctgctttgatgcagcactgtgatcctccacagaggcggtttccgttggagaagggggtatctccaccctggtggccctctggtaaagaggaatggtggggtcagttgggtctgccaaatgatcaagttcaacctccatacaagaagcctcatgatctgaagaaggcctggaaggttggtgttctgacggcggtaatcaaacacatctctcccgacattgctaagattcgcaagcttgttcgacagtcaaagtgcttgcaggataagatgacagctaaggagagtgctacttggcttgctattatcaatcaagaagaggctttggctcgtaagctgtatcctgacagctatccacagggatctctagctgttggtaatggttcctttttcatcagcgatgctagcgattacgatgtggaaggagtggataacgagagaaacaatgaagtggaatgtaaaccccatgacatcaatctccaaactggaattatgttacctaaagatagggttttgatgccaggtttagctccagtgaaaggagaaattattgatttaacttccgattttatccagaagaggaaggaaccatgttttgaggagtctgttgatcaaaagatatatacttgtgagtaccttcactgcccatacagcaattatcaagctggattccttgacaggacttcaagaaacaaccaccaaatgagttgtccattccggttcaattctgctcaaacacttactacacctaagtatcagatcaactatgagcacaacacagtttttcctgcacaaactgcaacttctaagccagcggtctcgtcagtcactgcttcctcttcgatgagtgcctcggggcttggactccctgaagatgatcagaggatcatttctgacctcataacatcatacgacaacaactttcagcaaaatggtagcatctgttccggaatttctgagattctagtaaaccaaagcctgcctcagcaacaaacagttgaacttcccatggatggcaacatcaacctaggacatatggagacctcagctcaagaaaccagcatgcctgtttatcgttcaacagagtttcaatatgatcaatgcaaaatgtcctttgacgcccccttcggtggaaacataaatgatataactgattacagattcggttccccgttcaacttgggaggaagcgactacgccgtggaacagctgacaaagcaggatatatctacatggtacctctgaactagtactagtattaaactgtcttatttcctatatgaaggcttgataggttgtatatgtttagataagtgatcaactctctgtcctttatatatacaggtattgtagtttctttgtgatgtagttgatgtttccatggttgtaagtgctaaacacaataattatctttatgcaattatgttttaaaaaaaaaaaaaaaaaaaaaaaaa

SEQ ID NO: 13 - DNA, 4758 bp, Arabidopsis thaliana
cttttctctctctatctctatctctcgtagcttgataagagtttctctcttttgaagatccgtttctctctctctcactgagactattgttgttaggtcaacttgcgatcatggcgatttcgaaggtctgaagctgatttcgaatggtttggagatatccgtagtggttaagcatatggaagtctatgttctgctcttggttgctctgttagggcttcctccatttggaccaacttagctgaatgttgtatgatctctctccttgaagcagcaaataagaagaaggtctggtccttaacttaacatctggttactagaggaaacttcagctattattaggtaaagaaagactgtacagagttgtataacaagtaagcgttagagtggctttgtttgcctcggtgatagaagaaccgactgattcgttgttgtgtgttagctttggagggaatcagatttcgcgagggaaggtgttttagatcaaatctgtgaattttactcaactgaggcttttagtgaaccacgactgtagagttgaccttgaatcctactctgagtaattatattatcagatagatttaggatggaagctgaaattgtgaatgtgagacctcagctagggtttatccagagaatggttcctgctctacttcctgtccttttggtttctgtcggatatattgatcccgggaaatgggttgcaaatatcgaaggaggtgctcgtttcgggtatgacttggtggcaattactctgcttttcaattttgccgccatcttatgccaatatgttgcagctcgcataagcgttgtgactggtaaacacttggctcagatctgcaatgaagaatatgacaagtggacgtgcatgttcttgggcattcaggcggagttctcagcaattctgctcgaccttaccatggttgtgggagttgcgcatgcacttaaccttttgtttggggtggagttatccactggagtgtttttggccgccatggatgcgtttttatttcctgttttcgcctctttccttgaaaatggtatggcaaatacagtatccatttactctgcaggcctggtattacttctctatgtatctggcgtcttgctgagtcagtctgagatcccactctctatgaatggagtgttaactcggttaaatggagagagcgcattcgcactgatgggtcttcttggcgcaagcatcgtccctcacaatttttatatccattcttattttgctggggaaagtacatcttcgtctgatgtcgacaagagcagcttgtgtcaagaccatttgttcgccatctttggtgtcttcagcggactgtcacttgtaaattatgtattgatgaatgcagcagctaatgtgtttcacagtactggccttgtggtactgacttttcacgatgccttgtcactaatggagcaggtatttatgagtccgctcattccagtggtctttttgatgctcttgttcttctctagtcaaattaccgcactagcttgggctttcggtggagaggtcgtcctgcatgacttcctgaagatagaaatacccgcttggcttcatcgtgctacaatcagaattcttgcagttgctcctgcgctttattgtgtatggacatctggtgcagacggaatataccagttacttatattcacccaggtcttggtggcaatgatgcttccttgctcggtaataccgcttttccgcattgcttcgtcgagacaaatcatgggtgtccataaaatccctcaggttggcgagttcctcgcacttacaacgtttttgggatttctggggttgaatgttgtttttgttgttgagatggtatttgggagcagtgactgggctggtggtttgagatggaataccgtgatgggcacctcgattcagtacaccactctgcttgtatcgtcatgtgcatccttatgcctgatactctggctggcagccacgccgctgaaatctgcgagtaacagagcggaagctcaaatatggaacatggatgctcaaaatgctttatcttatccatctgttcaagaagaggaaattgaaagaacagaaacaaggaggaacgaagacgaatcaatagtgcggttggaaagcagggtaaaggatcagttggatactacgtctgttactagctcggtctatgatttgccagagaacattctaatgacggatcaagaaatccgttcgagccctccagaggaaagagagttggatgtaaagtactctacctctcaagttagtagtcttaaggaagactctgatgtaaaggaacagtctgtattgcagtcaacagtggttaatgaggtcagtgataaggatctgattgttgaaacaaagatggcgaaaattgaaccaatgagtcctgtggagaagattgttagcatggagaataacagcaagtttattgaaaaggatgttgaaggggtttcatgggaaacagaagaagctaccaaagctgctcctacaagcaactttactgtcggatctgatggtcctccttcattccgcagcttaagtggggaagggggaagtgggactggaagcctttcacggttgcaaggtttgggacgtgctgcccggagacacttatctgcgatccttgatgaattttggggacatttatatgattttcatgggcaattggttgctgaagccagggcaaagaaactagatcagctgtttggcactgatcaaaagtcagcctcttctatgaaagcagattcgtttggaaaagacattagcagtggatattgcatgtcaccaactgcgaagggaatggattcacagatgacttcaagtttatatgattcactgaagcagcagaggacaccgggaagtatcgattcgttgtatggattacaaagaggttcgtcaccgtcaccgttggtcaaccgtatgcagatgttgggtgcatatggtaacaccactaataataataatgcttacgaattgagtgagagaagatactctagcctgcgtgctccatcatcttcagagggttgggaacaccaacaaccagctacagttcacggataccagatgaagtcatatgtagacaatttggcaaaagaaaggcttgaagccttacaatcccgtggagagatcccgacatcgagatctatggcgcttggtacattgagctatacacagcaacttgctttagccttgaaacagaagtcccagaatggtctaacccctggaccagctcctgggtttgagaattttgctgggtctagaagcatatcgcgacaatctgaaagatcttattacggtgttccatcttctggcaatactgatactgttggcgcagcagtagccaatgagaaaaaatatagtagcatgccagatatctcaggattgtctatgtccgcaaggaacatgcatttaccaaacaacaagagtggatactgggatccgtcaagtggaggaggagggtatggtgcgtcttatggtcggttaagcaatgaatcatcgttatattctaatttggggtcacgggtgggagtaccctcgacttatgatgacatttctcaatcaagaggaggctacagagatgcctacagtttgccacagagtgcaacaacagggaccggatcgctttggtccagacagccctttgagcagtttggtgtagcggagaggaatggtgctgttggtgaggagctcaggaatagatcgaatccgatcaatatagacaacaacgcttcttctaatgttgatgcagaggctaagcttcttcagtcgttcaggcactgtattctaaagcttattaaacttgaaggatccgagtggttgtttggacaaagcgatggagttgatgaagaactgattgaccgggtagctgcacgagagaagtttatctatgaagctgaagctcgagaaataaaccaggtgggtcacatgggggagccactaatttcatcggttcctaactgtggagatggttgcgtttggagagctgatttgattgtgagctttggagtttggtgcattcaccgtgtccttgacttgtctctcatggagagtcggcctgagctttggggaaagtacacttacgttctcaaccgcctacagggagtgattgatccggcgttctcaaagctgcggacaccaatgacaccgtgcttttgccttcagattccagcgagccaccagagagcgagtccgacttcagctaacggaatgttacctccggctgcaaaaccggctaaaggcaaatgcacaaccgcagtcacacttcttgatctaatcaaagacgttgaaatggcaatctcttgtagaaaaggccgaaccggtacagctgcaggtgatgtggctttcccaaaggggaaagagaatttggcttcggttttgaagcggtataaacgtcggttatcgaataaaccagtaggtatgaatcaggatggacccggttcaagaaaaaacgtgactgcgtacggatcattgggttgaagaagaagaacattgtgagaaatctcatgatcaaagtgacgtcgagagggaagccgaagaatcaaaactctcgcttttgattgctcctctgcttcgttaattgtgtattaagaaaagaagaaaaaaaatggatttttgttgcttcagaatttttcgctctttttttcttaatttggttgtaatgttatgtttatatacatatatcatcatcataggaccatagctacaaaccgaatccggtttgtgtaattctatgcggaatcataaagaaatcgtcggtttgaaatgtt

SEQ ID NO: 14 - DNA, 6022 bp, Arabidopsis thaliana
aggtggcacgagcacccataaccttcagacctatagatacaaatatgtatgtatacgttttttatatataaatattttatataattgatttttcgatcttcttttatctctctctttcgatggaactgagctctttctctctttcctcttcttttctctctctatctctatctctcgtagcttgataagagtttctctcttttgaagatccgtttctctctctctcactgagactattgttgttaggtcaacttgcgatcatggcgatttcgaaggtgacttctttcaaaaaccctaatcctctgtttttttttttattttgctggggggctttgtacggactttcatgggtttttgtagcttttccctcggcttttgcgcaaatgagactttctgggttttttttccagctttttataatttcatcaggtggatcgaattcgtagtttcagcttagatctctctccctcttcattatctggactttccagacttggagttcttcgggattgttttcggtttctgggttttgttttaattgcgagatttaagcttttttcttttttactactgtacttggtttgtggttgaccttttttttccttgaagatctgaatgcgtagatcatacgggatctttgcatttttgttgcttttcgtcagcgttacgattcttttagcttcagtttagttgaaatttgtattttttttgagcttatcttctttttgttgctgcttcatactaagatcaattattgatttgtaatactactgtatctgaagattttcaccataaaaaaaaaattcaggtctgaagctgatttcgaatggtttggagatatccgtagtggttaagcatatggaagtctatgttctgctcttggttgctctgttagggcttcctccatttggaccaacttagctgaatgttgtatgatctctctccttgaagcagcaaataagaagaaggtctggtccttaacttaacatctggttactagaggaaacttcagctattattaggtaaagaaagactgtacagagttgtataacaagtaagcgttagagtggctttgtttgcctcggtgatagaagaaccgactgattcgttgttgtgtgttagctttggagggaatcagatttcgcgagggaaggtgttttagatcaaatctgtgaattttactcaactgaggcttttagtgaaccacgactgtagagttgaccttgaatcctactctgagtaattatattatcagatagatttaggatggaagctgaaattgtgaatgtgagacctcagctagggtttatccagagaatggttcctgctctacttcctgtccttttggtttctgtcggatatattgatcccgggaaatgggttgcaaatatcgaaggaggtgctcgtttcgggtatgacttggtggcaattactctgcttttcaattttgccgccatcttatgccaatatgttgcagctcgcataagcgttgtgactggtaaacacttggctcaggtaaacatttttctgatctctaaagaacaaactttttaaaataacaaactgggctctgtggttgtcttgtcactttctcaaagtggaattctactaaccaccttctctatttttctaacattttaatgttctttactgggacagatctgcaatgaagaatatgacaagtggacgtgcatgttcttgggcattcaggcggagttctcagcaattctgctcgaccttaccatggtagttacttacaatctttgctgttcttaatttttttattatgtgataaaattttgattcctctgacttgagcttctctattataaacaggttgtgggagttgcgcatgcacttaaccttttgtttggggtggagttatccactggagtgtttttggccgccatggatgcgtttttatttcctgttttcgcctctttccttgtatgactggtcttcctgtcttgttttttttctccacgttcttgaaatagcattattggaaattagctgacatgcatacaatttctgacaggaaaatggtatggcaaatacagtatccatttactctgcaggcctggtattacttctctatgtatctggcgtcttgctgagtcagtctgagatcccactctctatgaatggagtgttaactcggttaaatggagagagcgcattcgcactgatgggtcttcttggcgcaagcatcgtccctcacaatttttatatccattcttattttgctggggtaccttttttctctttatatgtatctctcttttctgttaagaagcaataattatactaagcagtgaacgctctattacaggaaagtacatcttcgtctgatgtcgacaagagcagcttgtgtcaagaccatttgttcgccatctttggtgtcttcagcggactgtcacttgtaaattatgtattgatgaatgcagcagctaatgtgtttcacagtactggccttgtggtactgacttttcacgatgccttgtcactaatggagcaggtttgttctgacggttttatgttcgtattagtctataattcatttttagggaaaatgttcagaaatctctcgtgattattaattatcttgttcttgattgttgatcacaggtatttatgagtccgctcattccagtggtctttttgatgctcttgttcttctctagtcaaattaccgcactagcttgggctttcggtggagaggtcgtcctgcatgacttcctgaagatagaaatacccgcttggcttcatcgtgctacaatcagaattcttgcagttgctcctgcgctttattgtgtatggacatctggtgcagacggaatataccagttacttatattcacccaggtcttggtggcaatgatgcttccttgctcggtaataccgcttttccgcattgcttcgtcgagacaaatcatgggtgtccataaaatccctcaggttggcgagttcctcgcacttacaacgtttttgggatttctggggttgaatgttgtttttgttgttgagatggtatttgggagcagtgactgggctggtggtttgagatggaataccgtgatgggcacctcgattcagtacaccactctgcttgtatcgtcatgtgcatccttatgcctgatactctggctggcagccacgccgctgaaatctgcgagtaacagagcggaagctcaaatatggaacatggatgctcaaaatgctttatcttatccatctgttcaagaagaggaaattgaaagaacagaaacaaggaggaacgaagacgaatcaatagtgcggttggaaagcagggtaaaggatcagttggatactacgtctgttactagctcggtctatgatttgccagagaacattctaatgacggatcaagaaatccgttcgagccctccagaggaaagagagttggatgtaaagtactctacctctcaagttagtagtcttaaggaagactctgatgtaaaggaacagtctgtattgcagtcaacagtggttaatgaggtcagtgataaggatctgattgttgaaacaaagatggcgaaaattgaaccaatgagtcctgtggagaagattgttagcatggagaataacagcaagtttattgaaaaggatgttgaaggggtttcatgggaaacagaagaagctaccaaagctgctcctacaagcaactttactgtcggatctgatggtcctccttcattccgcagcttaagtggggaagggggaagtgggactggaagcctttcacggttgcaaggtttgggacgtgctgcccggagacacttatctgcgatccttgatgaattttggggacatttatatgattttcatgggcaattggttgctgaagccagggcaaagaaactagatcagctgtttggcactgatcaaaagtcagcctcttctatgaaagcagattcgtttggaaaagacattagcagtggatattgcatgtcaccaactgcgaagggaatggattcacagatgacttcaagtttatatgattcactgaagcagcagaggacaccgggaagtatcgattcgttgtatggattacaaagaggttcgtcaccgtcaccgttggtcaaccgtatgcagatgttgggtgcatatggtaacaccactaataataataatgcttacgaattgagtgagagaagatactctagcctgcgtgctccatcatcttcagagggttgggaacaccaacaaccagctacagttcacggataccagatgaagtcatatgtagacaatttggcaaaagaaaggcttgaagccttacaatcccgtggagagatcccgacatcgagatctatggcgcttggtacattgagctatacacagcaacttgctttagccttgaaacagaagtcccagaatggtctaacccctggaccagctcctgggtttgagaattttgctgggtctagaagcatatcgcgacaatctgaaagatcttattacggtgttccatcttctggcaatactgatactgttggcgcagcagtagccaatgagaaaaaatatagtagcatgccagatatctcaggattgtctatgtccgcaaggaacatgcatttaccaaacaacaagagtggatactgggatccgtcaagtggaggaggagggtatggtgcgtcttatggtcggttaagcaatgaatcatcgttatattctaatttggggtcacgggtgggagtaccctcgacttatgatgacatttctcaatcaagaggaggctacagagatgcctacagtttgccacagagtgcaacaacagggaccggatcgctttggtccagacagccctttgagcagtttggtgtagcggagaggaatggtgctgttggtgaggagctcaggaatagatcgaatccgatcaatatagacaacaacgcttcttctaatgttgatgcagaggctaagcttcttcagtcgttcaggcactgtattctaaagcttattaaacttgaaggatccgagtggttgtttggacaaagcgatggagttgatgaagaactgattgaccgggtagctgcacgagagaagtttatctatgaagctgaagctcgagaaataaaccaggtgggtcacatgggggagccactaatttcatcggttcctaactgtggagatggttgcgtttggagagctgatttgattgtgagctttggagtttggtgcattcaccgtgtccttgacttgtctctcatggagagtcggcctgagctttggggaaagtacacttacgttctcaaccgcctacaggtaacaaaaaccgcagtagttcattgaaaatcacagttttgcagtttgaaaatattgacatgtatggatttaaacagggagtgattgatccggcgttctcaaagctgcggacaccaatgacaccgtgcttttgccttcagattccagcgagccaccagagagcgagtccgacttcagctaacggaatgttacctccggctgcaaaaccggctaaaggcaaatgcacaaccgcagtcacacttcttgatctaatcaaagacgttgaaatggcaatctcttgtagaaaaggccgaaccggtacagctgcaggtgatgtggctttcccaaaggggaaagagaatttggcttcggttttgaagcggtataaacgtcggttatcgaataaaccagtaggtatgaatcaggatggacccggttcaagaaaaaacgtgactgcgtacggatcattgggttgaagaagaagaacattgtgagaaatctcatgatcaaagtgacgtcgagagggaagccgaagaatcaaaactctcgcttttgattgctcctctgcttcgttaattgtgtattaagaaaagaagaaaaaaaatggatttttgttgcttcagaatttttcgctctttttttcttaatttggttgtaatgttatgtttatatacatatatcatcatcataggaccatagctacaaaccgaatccggtttgtgtaattctatgcggaatcataaagaaatcgtcg

SEQ ID NO: 15 - DNA, 2413 bp, Arabidopsis thaliana
acagagagactccacaaagaaacgcaaataaacaaaagtcgctttctagccacgtgatctttcgtcgacttttcttcttcttcttcttcttcctcttcctcatctcgtatctctaacttttgtcgaagttcttttgatgaaactagggtttattatcttctccttctttttcccatcaccatagaaaaggcagagacctttttcttcatcatttttattctccttcttcttctgctgttcatttctccaggttacaatgatgtttaatgagatgggaatgtgtggaaacatggatttcttctcttctggatcacttggtgaagttgatttctgtcctgttccacaagctgagcctgattccattgttgaagatgactatactgatgatgagattgatgttgatgaattggagaggaggatgtggagagacaaaatgcggcttaaacgtctcaaggagcaggataagggtaaagaaggtgttgatgctgctaaacagaggcagtctcaagagcaagctaggaggaagaaaatgtctagagctcaagatgggatcttgaagtatatgttgaagatgatggaagtttgtaaagctcaaggctttgtttatgggattattccggagaatgggaagcctgtgactggtgcttctgataatttaagggagtggtggaaagataaggttaggtttgatcgtaatggtcctgcggctattaccaagtatcaagcggagaataatatcccggggattcatgaaggtaataacccgattggaccgactcctcataccttgcaagagcttcaagacacgactcttggatcgcttttgtctgcgttgatgcaacactgtgatcctcctcagagacgttttcctttggagaaaggagttcctcctccgtggtggcctaatgggaaagaggattggtggcctcaacttggtttgcctaaagatcaaggtcctgcaccttacaagaagcctcatgatttgaagaaggcgtggaaagtcggcgttttgactgcggttatcaagcatatgtttcctgatattgctaagatccgtaagctcgtgaggcaatctaaatgtttgcaggataagatgactgctaaagagagtgctacctggcttgctattattaaccaagaagagtccttggctagagagctttatcccgagtcatgtccacctctttctctgtctggtggaagttgctcgcttctgatgaatgattgcagtcaatacgatgttgaaggtttcgagaaggagtctcactatgaagtggaagagctcaagccagaaaaagttatgaattcttcaaactttgggatggttgctaaaatgcatgactttcctgtcaaagaagaagtcccagcaggaaactcggaattcatgagaaagagaaagccaaacagagatctgaacactattatggacagaaccgttttcacctgcgagaatcttgggtgtgcgcacagcgaaatcagccggggatttctggataggaattcgagagacaaccatcaactggcatgtccacatcgagacagtcgcttaccgtatggagcagcaccatccaggtttcatgtcaatgaagttaagcctgtagttggatttcctcagccaaggccagtgaactcagtagcccaaccaattgacttaacgggtatagttcctgaagatggacagaagatgatctcagagctcatgtccatgtacgacagaaatgtccagagcaaccaaacctctatggtcatggaaaatcaaagcgtgtcactgcttcaacccacagtccataaccatcaagaacatctccagttcccaggaaacatggtggaaggaagtttctttgaagacttgaacatcccaaacagagcaaacaacaacaacagcagcaacaatcaaacgttttttcaagggaacaacaacaacaacaatgtgtttaagttcgacactgcagatcacaacaactttgaagctgcacataacaacaacaataacagtagcggcaacaggttccagcttgtgtttgattccacaccgttcgacatggcgtcattcgattacagagatgatatgtcgatgccaggagtagtaggaacgatggatggaatgcagcagaagcagcaagatgtatccatatggttctaaagtcttggtagtagatttcatcttctcttatttttatcttttgtgttcttacattcactcaaccatgtaatattttttcctgggtctctctgtctctatcgcttgttatgatgtgtctgtaagagtctctaaaaactctctgttactgtgtgtctttgtctcggcttggtgaatctctctgtcatcatcagcttttagttacacacccgacttggggatgaacgaacactaaatgtaagttttcataatataaatatatttgcaagctct

SEQ ID NO: 16 - DNA, 2310 bp, Arabidopsis thaliana
tcttcttcttcttcctcttcctcatctcgtatctctaacttttgtcgaagttcttttgatgaaactagggtttattatcttctccttctttttcccatcaccatagaaaaggcagagacctttttcttcatcatttttattctccttcttcttctgctgttcatttctccaggttacaatgatgtttaatgagatgggaatgtgtggaaacatggatttcttctcttctggatcacttggtgaagttgatttctgtcctgttccacaagctgagcctgattccattgttgaagatgactatactgatgatgagattgatgttgatgaattggagaggaggatgtggagagacaaaatgcggcttaaacgtctcaaggagcaggataagggtaaagaaggtgttgatgctgctaaacagaggcagtctcaagagcaagctaggaggaagaaaatgtctagagctcaagatgggatcttgaagtatatgttgaagatgatggaagtttgtaaagctcaaggctttgtttatgggattattccggagaatgggaagcctgtgactggtgcttctgataatttaagggagtggtggaaagataaggttaggtttgatcgtaatggtcctgcggctattaccaagtatcaagcggagaataatatcccggggattcatgaaggtaataacccgattggaccgactcctcataccttgcaagagcttcaagacacgactcttggatcgcttttgtctgcgttgatgcaacactgtgatcctcctcagagacgttttcctttggagaaaggagttcctcctccgtggtggcctaatgggaaagaggattggtggcctcaacttggtttgcctaaagatcaaggtcctgcaccttacaagaagcctcatgatttgaagaaggcgtggaaagtcggcgttttgactgcggttatcaagcatatgtttcctgatattgctaagatccgtaagctcgtgaggcaatctaaatgtttgcaggataagatgactgctaaagagagtgctacctggcttgctattattaaccaagaagagtccttggctagagagctttatcccgagtcatgtccacctctttctctgtctggtggaagttgctcgcttctgatgaatgattgcagtcaatacgatgttgaaggtttcgagaaggagtctcactatgaagtggaagagctcaagccagaaaaagttatgaattcttcaaactttgggatggttgctaaaatgcatgactttcctgtcaaagaagaagtcccagcaggaaactcggaattcatgagaaagagaaagccaaacagagatctgaacactattatggacagaaccgttttcacctgcgagaatcttgggtgtgcgcacagcgaaatcagccggggatttctggataggaattcgagagacaaccatcaactggcatgtccacatcgagacagtcgcttaccgtatggagcagcaccatccaggtttcatgtcaatgaagttaagcctgtagttggatttcctcagccaaggccagtgaactcagtagcccaaccaattgacttaacgggtatagttcctgaagatggacagaagatgatctcagagctcatgtccatgtacgacagaaatgtccagagcaaccaaacctctatggtcatggaaaatcaaagcgtgtcactgcttcaacccacagtccataaccatcaagaacatctccagttcccaggaaacatggtggaaggaagtttctttgaagacttgaacatcccaaacagagcaaacaacaacaacagcagcaacaatcaaacgttttttcaagggaacaacaacaacaacaatgtgtttaagttcgacactgcagatcacaacaactttgaagctgcacataacaacaacaataacagtagcggcaacaggttccagcttgtgtttgattccacaccgttcgacatggcgtcattcgattacagagatgatatgtcgatgccaggagtagtaggaacgatggatggaatgcagcagaagcagcaagatgtatccatatggttctaaagtcttggtagtagatttcatcttctcttatttttatcttttgtgttcttacattcactcaaccatgtaatattttttcctgggtctctctgtctctatcgcttgttatgatgtgtctgtaagagtctctaaaaactctctgttactgtgtgtctttgtctcggcttggtgaatctctctgtcatcatcagcttttagttacacacccgacttggggatgaacgaacactaaatgtaagttttca